(19) United States
(12) Patent Application Publication
(10) Pub. No.: US 2015/0252148 A1
(43) Pub. Date: Sep. 10, 2015

(54) SYNTHETICALLY DESIGNED EXTRACELLULAR MICROENVIRONMENT

(71) Applicant: KOLLODIS BIOSCIENCE, CO., LTD., Incheon (KR)

(72) Inventors: Seung Goo Lee, Pohang-si (KR); Hyo Jin Bong, Seoul (KR); Bong Jin Hong, Pohang-si (KR); Kil Won Cho, Pohang-si (KR); Sangjae Lee, Seoul (KR)

(73) Assignee: KOLLODISBIOSCIENCE, CO., LTD., Incheon (KR)

(21) Appl. No.: 14/427,873

(22) PCT Filed: Sep. 13, 2013
(86) PCT No.: PCT/KR2013/008306

§ 371 Date: Mar. 12, 2015

(30) Foreign Application Priority Data
Sep. 13, 2012 (KR) 10-2012-0101746

(51) Int. Cl. C08G 81/00; A61L 27/24; C12N 5/00; A61L 27/54; A61L 27/56; A61L 27/22; A61L 27/52

(52) U.S. Cl. CPC C08G 81/00; A61L 27/227; A61L 27/24; A61L 27/52; A61L 27/54; A61L 27/56; C12N 5/0068; A61L 2430/00; A61L 2300/252; A61L 2300/412; C08G 2210/00; C12N 2533/30; C12N 2533/52; C12N 2533/54

(57) ABSTRACT

The present invention provides for a biochemically and physically defined extracellular microenvironment prepared from mussel adhesive proteins recombinantly functionalized with a variety of bioactive peptides such as extracellular matrix-derived or growth factor-derived peptides. The synthetic extracellular microenvironment can be customized to regulate cellular behavior such as cell adhesion, growth, differentiation and morphogenesis in a variety of cells. The invention provides for a modulatory extracellular microenvironment by presenting a matricryptic site into said mussel adhesive proteins. The invention also provides for devices and methods for screening for optimal combinations of ECM derived peptide motifs in order to create a microenvironment that can regulate specific cellular behavior.

### TECHNICAL FIELD

The present invention is directed to synthetic modulatory microenvironments that mimic biochemically and/or mechanically natural ECM microenvironments.

The present application claims priority to and the benefit of Korean patent application (KR 10-2012-0101746), filed Sep. 13, 2012 which is hereby incorporated by reference for all purposes as if fully set forth herein.

### BACKGROUND ART

Cellular microenvironments, defined by biochemical cues and physical cues, are a deciding factor in a wide range of cellular processes including cell adhesion, proliferation, differentiation, and expression of phenotype-specific functions (See Discher D E, et al., Science. 2009, 26; 324 (5935):1673-7 and Hynes R O, Trends Cell Biol.; 1999, 9(12):M33-7).

It is well recognized that cells generally interact with their surrounding microenvironment in order to survive and biologically function; or, in order to determine their direction of differentiation (Song X, et al., Science. 2002; 296: 1855-1857 and Li L, et al., Annu Rev Cell Dev Biol. 2005; 21: 605-631).

Most cells in tissues are surrounded on all sides by a complex set of extracellular matrix (ECM) proteins that are critical in guiding cell function. Cells bind to the ECM via specific cell surface receptors such as integrin receptors, and this binding serves as a biochemical cue that can directly affect cell function. In addition, the ECM acts as a modulator of biochemical and mechanical stimuli that are present in tissues. For example, ECM proteins can sequester and release growth factors, control the rate of nutrient supply, as well as control cell shape and transmit mechanical signals to the cell surface.

ECM and growth factor signaling environments are the important mechanisms for regulating cell fate; and, these microenvironmental stimuli are processed through combinatorial signaling pathways. The interactions between signaling pathways are critical in determining cell fate. (Flaim C J, et al., Stem Cells Dev. 2008, 17(1):29-39).

For example, fibroblast proliferation, differentiation into myofibroblasts, and increased collagen synthesis are key events during both normal wound repair- and the fibroblast proliferation and differentiation are controlled by combinatorial signaling pathways (Grotendorst G R, et al., FASEB J. 2004 18(3): 469-79).

The mechanical compliance of the ECM that surrounds cells is also an important factor in controlling cell function in both 2D and 3D microenviornment. For example of MSC cell fate, softer substrates ranging 0.1 to 1 kPa tend to guide MSCs down neurogenic, adipogenic and chondrogenic pathways, while stiffer substrates than 10 kPa have been shown to support myogenesis and osteogenesis depending on the specific composition of the culture media (Engler et al. Matrix elasticity directs stem cell lineage specification. Cell 2006, 126:677-689; Park J S, et al., (2011) The effect of matrix stiffness on the differentiation of mesenchymal stem cells in response to TGF-β. Biomaterials 32: 3921-3930.), although the underlying mechanisms by which stem cells sense and respond to substrate stiffness is not fully understood. Time-dependent changes in matrix elasticity also played a key role in directing stem cell fate (Guvendiren M, Burdick J A (2012) Stiffening hydrogels to probe short and long-term cellular responses to dynamic mechanics. Nat Commun 3:792; Young J L, Engler A J (2011) Hydrogels with time-dependent material properties enhance cardiomyocyte differentiation in vitro. Biomaterials 32:1002-1009). Interestingly, apoptosis also seems to be regulated by matrix stiffness (Pelling et al. (2009) Mechanical dynamics of single cells during early apoptosis. Cell Motil Cytoskeleton 66:409-422).

Therefore, the ultimate fate of a cell to proliferate, differentiate, migrate, apoptosis or perform other specific functions is a coordinated response to the molecular interactions with these ECM microenvironmental effectors (Lutolf M P, et al., Nat Biotechnol. 2005, 23(1):47-55).

Many attempts have been made to create a synthetic extracellular microenvironment by incorporating cell adhesion ligands into biomaterials. Both biologically derived or synthetic materials have been explored as an extracellular microenvironment to gain control over the material and thus over the cellular behavior they induced. One example is a crosslinkable hyaluronic acid, alginate or polyethylene glycol based hydrogel with an RGD peptide motif grafted onto the polymer backbone. (Woerly et al., J. Neural Transplant. Plasticity, 1995, 5:245-255.; Imen et al., Biomaterials, 2006, 27, p3451-3458; U.S. Pat. No. 20060134050).

Complexities associated with native extracellular matrix proteins, including complex structural composition, purification, immunogenicity and pathogen transmission have driven the development of synthetic biomaterials for use as 2D or 3D extracellular microenvironments in order to mimic the regulatory characteristics of natural ECMs and ECM-bound growth factors [Lutolf M P, et al., Nat Biotechnol. 23(1):47-55 (2005) and Ogiwara K, et al., Biotechnol Lett. 27(20):1633-7 (2005)].

However, existing technologies do not create a microenvironment that induces a combinatorial signal pathway by simultaneously activating at least two different cell surface receptors, due to their lack of physical or biochemical attributes. In addition, various microenvironmental cues are often intertwined and cannot be individually controlled in existing technologies. For example, type I collagen based hydrogel has been widely used as 3D scaffold, but increasing its concentration to increase biochemical ligand density lead to simultaneous change in the mechanical stiffness of the matrix (Wakitani S, et al., Repair of large full-thickness articular cartilage defects with allograft articular chondrocytes embedded in a collagen gel. Tissue Eng A 1998; 4, 429-44; Sumanasinghe R D, et al., Osteogenic differentiation of human mesenchymal stem cells in collagen matrices: effect of uniaxial cyclic tensile strain on bone morphogenetic protein (BMP-2) mRNA expression. Tissue Eng A 2006; 12:3459-65).

We have developed a biochemically and physically defined synthetic microenvironment that mimics native extracellular microenvironments by presenting combinatorial receptor-ligand interactions, controlled pore size and elasticity of a synthetic matrix. Our synthetic microenvironment can be used as an array of cell culture environments for screening of cell culture or tissue engineering environment by elucidating or regulating cellular behaviors such as cell adhesion, migration, growth, proliferation or morphogenesis as evidenced in cell adhesion and endothelial tube formation assays.

### DISCLOSURE OF INVENTION

### Technical Problem

The present invention is directed to synthetic modulatory microenvironments that mimic biochemically and/or mechanically natural ECM microenvironments.

### Solution to Problem

The present invention provides a synthetic microenvironment comprised of a crosslinkable biomaterial composition presenting at least one or more ECM-derived or growth factor derived peptide motifs that precisely regulate cellular behavior such as cell adhesion, migration, growth or differentiation.

In accordance with one aspect of the invention, there is provided a crosslinkable biomaterial composition for a synthetic 3D microenvironment created in situ, comprised of a biomaterial functionalized with at least one or more extracellular matrix (ECM)- or growth factor (GF)-derived peptide motifs and a crosslinking agent, wherein said crosslinking agent mediates its crosslinking function chemically via covalent, ionic, hydrogen-bonded, and Van der Waals interactions or, physically via molecular entanglement and intertwining or both chemical and physical crosslinking under a wide range of pH conditions.

In one embodiment of the present invention, there is a crosslinkable biomaterial composition for a synthetic 3D microenviornment comprised of a recombinant protein functionalized with at least one or more peptide motifs derived from a variety of extracellular matrix proteins or growth factors, and a crosslinking agent, wherein said crosslinking agent mediates its crosslinking function chemically via crosslinking under a wide range of pH conditions.

Any suitable recombinant protein including but not limited to fibrin, elastin, mussel adhesive protein may be used as said protein. Preferably, said protein is a recombinant mussel adhesive protein.

Any suitable mussel adhesive protein may be used as the biomaterial in this invention. The biomaterial compositions that generate a microenvironment are basically composed of two components. The first component is a mussel adhesive protein functionalized with bioactive peptides. The second component is a crosslinkable agent. Both components are commercially available materials or are obtained from synthetic or natural sources. Examples of commercially available proteins include MAPTrix™ ECM marketed by Kollodis BioSciences, Inc. (North Augusta, S.C.). An optional third component is a biocompatible polymer (e.g., polyethylene glycol or polyvinylalcohol), which may be added to the compositions to enhance their physicomechanical characteristics such as physical or mechanical properties of a customizable microenvironment.

The MAPTrix™ ECMs, developed by Kollodis BioSciences Inc., are predesigned mussel adhesive protein-based ECM mimetics. The mussel adhesive proteins were recombinantly fuctionalized with a variety of ECMs- or GFs-derived peptides in order to mimic the bioactivity of naturally occurring ECMs or GFs, which were demonstrated to have a similar bioactivity to natural or recombinant ECMs or GFs in primary cell cultures as compared to natural or recombinant ECM proteins or GF proteins. The pre-designed MAPTrix™ ECM mimetics are highly advantageous for creating extracellular microenvironments. For example, it provides for the design of cell-specific or user-defined regulation of extracellular microenvironments to emulate the native microenvironment in terms of biochemical cues.

The MAPTrix™ ECM is a mussel adhesive protein recombinantly functionalized with bioactive peptides, a fusion protein comprising a first peptide of mussel foot protein FP-5 (SEQ ID NO: 2) that is selected from the group consisting SEQ ID NOs: 10-13 and a second peptide of at least one selected from the group consisting of mussel FP-1 selected from the group consisting of SEQ ID Nos: 1-3, mussel FP-2 (SEQ ID NO: 4), mussel FP-3 selected from the group consisting of SEQ ID Nos: 5-8, mussel FP-4 (SEQ ID NO: 9), mussel FP-6 (SEQ ID NO: 14) and fragment thereof, and the second peptide is linked to C-terminus, N-terminus or C- and N-terminus of the FP-5. Preferably, the second peptide is The FP-1 comprising an amino acid sequence of SEQ ID NO: 1.

Bioactive peptides are necessary for the present invention in order to mimic the microenvironments of a natural extracellular matrix. Additional components such as growth factors, for example, fibroblast growth factor (FGF), transforming growth factor (TGF), epidermal growth factor (EGF), platelet-derived growth factor (PDGF), nerve growth factor (NGF), vascular endothelial growth factor (VEGF), or substance P, may also be included to further enhance the beneficial effect of the extracellular environment mimic on cell and tissue culture, medical devices and treatments, or for other related applications.

Bioactive peptides are natural or synthetic peptides derived from ECM proteins or growth factors in order to emulate the biochemical or biophysical cues of a natural extracellular microenvironment. The ECM proteins can be fibrous proteins such as collagens, fibronectin, laminin, vitronectin, growth factors, and the like. ECM proteins can influence activity of adhesion receptor such as integrin directly, and in turn, adhesion receptor such as integrins may activate signaling pathways by coclustering with kinases and adaptor proteins in focal adhesion complexes after their association with polyvalent extracellular matrix (ECM) proteins. For example, a RGD containing peptide segment from fibronectin, laminin or vitronectin to integrins may regulate to its integrin activity.

A suitable combination of peptide motifs-from ECM proteins that together create an extracellular microenvironment in order to induce combinatorial signaling are selected from ECM proteins or growth factors. Said ECM proteins are selected from collagen, fibronectin, laminin, vitronectin, heparin-binding domain, entactin, or fibrinogen. For example, mixtures of MAPTrix™ ECM containing GFPGER (SEQ ID NO: 22) that activates integrin α2β1, derived from collagen type I, and MAPTrix™ ECM containing IKVAV (SEQ ID NO: 37) that activates integrin αvβ3, derived from laminin can activate two different integrins αvβ3-αvβ1 at the same time, leading to endothelial tube formation. Said growth factors are selected from fibroblast growth factor, transforming growth factor, nerve growth factor, epidermal growth factor, VEGF, or PDGF.

Preferably, a suitable combination of peptide motifs has a formula A-B or A1-B1, wherein A is the peptide motif that activates integrin αvβ3, αvβ5, heparin, or syndecan, and B is the peptide motif that activates integrin α2β1, α3β1, α4β1, α5β1, or α6β1. A 1 is the peptide motif that activates growth factor receptors and B1 is the peptide motif that activates integrin, heparin, or syndecan.

More preferably, a suitable peptide motif (A) to activate integrin αvβ3, αvβ5 or syndecan is selected from IDAPS(SEQ ID:60), IKVAV(SEQ ID:37), RQVFQVAYIIIKA(SEQ ID:36), KAFDITYVRLKF(SEQ ID:47), MNYYSNS(SEQ ID:31), RGDV(SEQ ID:63), WQPPRARI(SEQ ID NO: 57), RKRLQVQLSIRT(SEQ ID NO: 40), KNSFMALYLSKG(SEQ ID NO: 41), SPPRRARVT(SEQ ID NO: 56), KNNQKSEPLIGRKKT(SEQ ID NO: 58), GDLGRPGRKGRPGPP(SEQ ID NO: 98), ATETTITISWRTKTE(SEQ ID NO: 99), TLFLAHGRLVFM(SEQ ID NO: 100), KGHRGF(SEQ ID NO: 21), FRHRNRKGY(SEQ ID NO: 101), KRSR(SEQ ID NO: 102), FHRRIKA(SEQ ID NO: 103), HAV(SEQ ID NO: 104), ADTPPV(SEQ ID NO: 105), DQNDN(SEQ ID NO: 106). Another suitable peptide motif (B) to activate activates integrin α1β1, α2β1, α3β1, α4β1, α5β1, or α6β1 is selected from the following Table 1.

TABLE 1
Bioactive peptide motif and its receptor

| Receptor | Peptide motif | ECM Type |
|---|---|---|
| integrin | GLPGER(SEQ IDNO: 20) | collagen |
| α1β1 or α2β1 | KGHRGF(SEQ ID NO: 21) | |
| | GFPGER(SEQ ID NO: 22) | |
| | DEGA(SEQ ID NO: 23) | |
| | GTPGPQGIAGQRGVV(SEQ ID NO: 24) | |
| | GLSGER(SEQ ID NO: 25) | |
| | GASGER(SEQ ID NO: 26) | |
| | GAPGER(SEQ ID NO: 27) | |
| | TAGSCLRKFSTM(SEQ ID NO: 28) | |
| | GEFYFDLRLKGDK(SEQ ID NO: 29) | |
| | TAIPSCPEGTVPLYS(SEQ ID NO: 30) | |
| | MNYYSNS(SEQ ID NO: 31) | |
| | ISRCQVCMKKRH(SEQ ID NO: 32) | |
| | GLKGEN(SEQ ID NO: 33) | |
| | GLPGEN(SEQ ID NO: 34) | |
| | GLPGEA(SEQ ID NO: 35) | |
| integrin | RQVFQVAYIIIKA(SEQ ID NO: 36) | laminin |
| α3β1 or α6β1 | IKVAV(SEQ ID NO: 37) | |
| | NRWHSIYITRFG(SEQ ID NO: 38) | |
| | TWYKIAFQRNRK(SEQ ID NO: 39) | |
| | RKRLQVQLSIRT(SEQ ID NO: 40) | |
| | KNSFMALYLSKG(SEQ ID NO: 41) | |
| | DYATLQLQEGRLHFMFDLG(SEQ ID NO: 42) | |
| | GIIFFL(SEQ ID NO: 43) | |
| | YIGSR(SEQ ID NO: 44) | |
| | RYVVLPR(SEQ ID NO: 45) | |
| | PDSGR(SEQ ID NO: 46) | |
| | KAFDITYVRLKF(SEQ ID NO: 47) | |
| | RNIAEIIKDI (SEQ ID NO: 48) | |
| integrin | KLDAPT (SEQ ID NO: 49) | fibronectin |
| α4β1 or α5β1 | PHSRN (SEQ ID NO: 50) | |
| | RGD (SEQ ID NO: 51) | |
| | GRGDSP (SEQ ID NO: 52) | |
| | PHSRNSGSGSGSGSGRGDSP(SEQ ID NO: 53) | |
| | YRVRVTPKEKTGPMKE(SEQ ID NO: 54) | |
| | EDGIHEL(SEQ ID NO: 55) | |
| | SPPRRARVT(SEQ ID NO: 56) | |
| | WQPPRARI(SEQ ID NO: 57) | |
| | KNNQKSEPLIGRKKT(SEQ ID NO: 58) | |
| | EILDVPST(SEQ ID NO: 59) | |
| | IDAPS(SEQ ID NO: 60) | |
| | REDV(SEQ ID NO: 61) | |
| | LEDV(SEQ ID NO: 62) | |
| Fibroblast | TGQYLAMDTDGLLYGS (SEQ ID NO: 91) | FGF-1 |
| growth factor | WFVGLKKNGSCKRG (SEQ ID NO: 92) | |
| receptor | HFKDPKRLYCK (SEQ ID NO: 93) | FGF-2 |
| | FLPMSAKS (SEQ ID NO: 94) | |
| | KTGPGQKAIL (SEQ ID NO: 95) | |
| | ANRYLAMKEDGRLLAS (SEQ ID NO: 96) | |
| | WYVALKRTGQYKLG (SEQ ID NO: 97) | |
| | SGRYLAMNKRGRLYAS (SEQ ID NO: 107) | FGF-3 |
| | SGLYLGMNEKGELYGS(SEQ ID NO: 108) | FGF-9 |
| | SNYYLAMNKKGKLYGS (SEQ ID NO: 109) | FGF-10 |
| | SEKYICMNKRGKLIGK (SEQ ID NO: 110) | FGF-17 |
| TGF receptor | HADLLAVVAASQ (SEQ ID NO: 111) | TGF α |
| | KVLALYNK (SEQ ID NO: 112) | TGF β |
| EGF receptor | CMHIESLDSYTC (SEQ ID NO: 113) | EGF |
| NGF receptor | PEAHWTKLQHSLDTALR (SEQ ID NO: 114) | NGF |
| Heparin | GDLGRPGRKGRPGPP (SEQ ID NO: 98) | Collagen |
| | ATETTITISWRTKTE (SEQ ID NO: 99) | Fibronectin |
| | TLFLAHGRLVFM (SEQ ID NO: 100) | Laminin |
| | FRHRNRKGY (SEQ ID NO: 101) | Vitronectin |
| | KRSR (SEQ ID NO: 102) | Bone |
| | | sialoprotein |
| PDGF receptor | SVLYTAVQPNE(SEQ ID NO: 115) | PDGF |
| VEGF receptor | KLTWQELYQLKYKGI (SEQ ID NO: 116) | VEGF |

In one embodiment of the present invention, a synthetic microenvironment that combinatorially regulates the activity of both integrin αv subtype and integrin β subtype is provided. The mussel adhesive protein is a combination of functional mussel adhesive proteins, mainly composed of mussel adhesive protein functionalized with a peptide such as collagen type I derived peptide GFPGER (SEQ ID NO: 22) to target α2β1 and a peptide such as laminin-derived peptide IKVAV (SEQ ID NO: 37) to target αvβ3.

In one embodiment of the present invention, a synthetic microenvironment that combinatorially regulates the activity of both integrin α subtype, or its subtype thereof, and integrin β is provided. The mussel adhesive protein is a combination of functional mussel adhesive proteins, mainly composed of mussel adhesive protein functionalized with a peptide such as collagen type I derived peptide GFPGER (SEQ ID NO: 22) to target α2β1 and a peptide such as fibronectin-derived peptide GRGDSP (SEQ ID NO: 52) to target α5β1.

In one embodiment of the present invention, a synthetic microenvironment that combinatorially regulates the activity of both integrin, or its subtype thereof, and heparin is provided. The mussel adhesive protein is a combination of functional mussel adhesive proteins, mainly composed of mussel adhesive protein functionalized with a peptide such as collagen type I derived peptide GFPGER (SEQ ID NO: 22) to target α2β1 and a peptide such as collagen type I derived peptide KGHRGF (SEQ ID NO: 22) to target heparin.

In one embodiment of the present invention, a synthetic microenvironment that combinatorially regulates the activity of both integrin, or its subtype thereof, and growth factor receptor is provided. The mussel adhesive protein is a combination of functional mussel adhesive proteins, mainly composed of mussel adhesive protein functionalized with a peptide such as fibronectin derived peptide GRGDSP (SEQ ID NO: 52) to target α5β1 and a peptide such as FGF-derived peptide GRGDSP(SEQ ID NO: 52) to target FGF receptor; FGFR2IIIc.

In another embodiment of the present invention, the mussel adhesive protein is a fusion protein of FP-151 which was recombinantly functionalized with fibronectin-derived peptide GRGDSP (SEQ ID NO: 52) to form a fibronectin rich extracellular matrix mimetic hydrogel.

A chemically crosslinkable agent suitable for use in this invention can be any biocompatible polymer, of natural or synthetic origin. Preferably, a crosslinkable agent is a synthetic polymer which has the appropriate functional groups such that it can be covalently linked directly or through a linker to a mussel adhesive protein. Any polymer meeting the above requirements is useful herein, and the selection of the specific polymer and acquisitions or preparation of such polymer would be conventionally practiced in the art (See The Biomedical Engineering Handbook, ed. Bronzino, Section 4, ed. Park.). Preferred for such crosslinkable polymers are selected from groups comprising poly(alkylene oxides) particularly poly(ethylene glycols), poly(vinyl alcohols), polypeptides, poly(amino acids), such as poly(lysine), poly(allylamines) (PAM), poly(acrylates), polyesters, polyphosphazenes, pluronic polyols, polyoxamers, poly(uronic acids) and copolymers, including graft polymers thereof.

The polymer may be selected to have a wide range of molecular weights, generally from as low as 1,000 up to millions of Daltons. Preferably, the selected polymer has a molecular weight of less than about 30,000 to 50,000 or one in which the backbone of the polymer itself is degradable. Polymers with a degradable polymeric backbone section include those with a backbone having hydrolyzable groups therein, such as polymers containing ester groups in the backbone, for example, aliphatic polyesters of the poly(a-hydroxy acids) including poly(glycolic acid) and poly(lactic acid). When the backbone is itself degradable, it need not be of low molecular weight to provide such degradability.

In one embodiment of the present invention, a 3D extracellular matrix mimetic composition formed in situ is provided. The composition is comprised of multiple-arm PEG and mussel adhesive proteins that mimic the 3D ECM microenvironments of native ECM. Multiple-arm PEG can be selected from the group consisting of 4 arm, 6 arm, 8 arm or 10 arm PEG. Preferred multiple-arm PEG is one selected from the group consisting of 4 to 8 arm. The most preferred multiple arm PEG is 6 and 8 arm PEG.

In another preferred embodiment, a preferred compound is one selected from the group consisting of: 4 to 8-arm PEG-succinic acid, 4 to 8-arm PEG-glutaric acid, 4 to 8-arm PEG-succimidyl succinate, 4 to 8-arm PEG-succimidyl glutarate, 4 to 8-arm PEG-acrylate, or 4 to 8-arm PEG-propion aldehyde.

A synthetic microenviornment-forming composition comprised of the 8-arm PEG-SG can be readily formed with mussel adhesive proteins functionalized with ECM derived peptides; or, a hydrogel-forming composition comprised of the 6-arm PEG-SG can be formed with mussel adhesive proteins, or a mixture of mussel adhesive proteins with 6-arm PEG-amine etc. MAPTrix™ HyGel, formed from MAPTrix™ ECM and multi-arm PEG, used in the present invention was described in PCT/KR2011/001831 (Adhesive extracellular matrix mimic), incorporated herein by reference.

In one embodiment of the invention, a synthetic microenvironment for endothelial morphogenesis is provided which presents angiogenic integrin mediated combinatorial signaling.

Endothelial cells express a broad range of integrin subunits. Vascular endothelial cells express a subset of integrins including αvβ3, αvβ5, α1β1, α2β1, α3β1, α5β1, α6β1, α6β4 and these bind a combination of ligands.

α1β1, α3β1 and α5β1 are expressed at low levels in quiescent vessels but at least α5β1 is upregulated during angiogenesis (Kairbaan M, et al., Cell Tissue Res (2003) 314:131-144).

αvβ3, αvβ5 and α2β1 are barely detectable in quiescent vessels but their expression is elevated greatly in sprouts (Max et al., Eur J Cancer (1997) 33:208-208).

In one embodiment of the invention, a synthetic 3D microenvironment that regulates β1 integrin-containing heterodimers which were exploited by endothelial cells for cellular morphogenesis such as endothelial tube formation.

Integrins are a superfamily of cell-surface adhesion molecules formed from 18 different α chains (α1-α11, αv, α_{IIb}, α_{L}, α_{M}, α_{X}, α_{D}, α_{E}) and eight different β chains (β1-β8) that assemble non-covalently as heterodimers. Integrins play a major part in the mediation of cell-cell and cell-matrix interactions, and are implicated in major cellular functions such as cell growth, survival, differentiation, and migration.

In endothelial cells (EC), cell-matrix interactions mediated by some integrins such as αv are important modulators of cell morphogenesis (Stupack D G, et al., Curr Top Dev Biol. 2004, 64:207-38 Wickstrom S A, et al., Adv Cancer Res. 2005; 94:197-229.).

The αv integrin subunit partners selectively with four different β subunits (β3, β5, β6 and β8) and also with β1, which in turn can partner with a dozen other a subunits.

β1 integrin is needed for EC adhesion, migration and survival during angiogenesis (Carlson T R, et al., Development. 2008; 135(12):2193-202).

The β1 subunit can associate with at least 10 different a subunits forming the largest subfamily of integrins. Members of the β1 integrin subfamily primarily bind to components of the ECM such as fibronectin, collagens, and laminins, but some of them also participate in direct cell-cell adhesion (Hynes, 1992; Haas and Plow, 1994).

In one embodiment, a synthetic microenvironment for cellular morphogenesis is provided. The synthetic microenvironment is comprised of MAPTrix™ compositions that regulate β1 integrin-containing heterodimers which can be exploited by endothelial cells for morphogenesis. The MAPTrix™ composition suitable for this invention presents at least two different bioactive peptide motifs, whereas one peptide motif regulates αv containing integrin and the other one regulates β1 containing integrin. Preferably, β1 integrin-containing heterodimers is selected from α2β1 or α5β1. In a preferred embodiment, the MAPTrix™ composition simultaneously regulates α2β1 and αvβ3 integrins. In another preferred embodiment, the MAPTrix™ composition simultaneously regulates α5β1 and αvβ3 integrins.

The present invention also provides a modulus controlled microenvironment whereas its pore size is consistent by addition of an enhancer to the biomaterial composition.

An enhancer of the present invention physically intertwines molecular chains formed from crosslinking polymer between mussel adhesive protein and crosslinking agent to form interpenetrating chains. The resultant microenvironment can offer controlled elasticity.

An enhancer can be selected from among natural, semi-synthetic, or synthetic materials that are crosslinkable or non-crosslinkable.

An enhancer can be a polysaccharide, such as one or more selected from, including but not limited to, hyaluronic acid, alginate, chitins, chitosan and derivatives thereof, cellulose and derivatives thereof. Additionally, an enhancer can be a polypeptide or protein selected from, including but not limited to, collagen, fibrinogen, gelatin and derivatives thereof. As for semi-synthetic or synthetic polymer, poly(L-lysine), poly(glutamic acid), poly(aspartic acid) can be selected. A homo- or co-polymer comprised of a monomer selected from (meth)acrylamides, (meth)acrylic acid and salts thereof, (meth)acrylates, ethylene glycol, ethylene oxide, styrene sulfonates, vinyl acetate, or vincyl alcohol.

Preferred enhances are homo- or co-polymers of naturally occurring polysaccharides, including chitosan or chitins, synthetic polymer, such as poly(vinyl alcohol), poly(glutamic acid), poly(lactic acid).

In one embodiment, an elasticity controlled microenviornment-forming composition comprised of the multi-arm PEG-SG can be readily formed with mussel adhesive proteins functionalized with ECM derived peptides and an enhancer to increase elasticity of mussel adhesive protein-multi-arm PEG or, a hydrogel-forming composition comprised of the 6-arm PEG-SG can be formed with mussel adhesive proteins, or a mixture of mussel adhesive proteins with 6-arm PEG-amine etc.

In one embodiment, an elasticity controlled microenvironment-forming extracellular matrix mimetic composition formed in situ is provided. The composition is comprised of multiple-arm PEG, mussel adhesive protein containing GRGDSP, and an enhancer that mimic a native extracellular microenvironments. Multiple-arm PEG can be selected from the group consisting of 4 arm, 6 arm, 8 arm, 10 arm, or 12 arm PEG. Preferred multiple-arm PEG is one selected from the group consisting of 4 to 10 arm. The most preferred multiple arm PEG is 4, 6, and 8 arm PEG.

In another preferred embodiment, a preferred compound is one selected from the group consisting of: 4 to 8-arm PEG-succinic acid, 4 to 8-arm PEG-glutaric acid, 4 to 8-arm PEG-succimidyl succinate, 4 to 8-arm PEG-succimidyl glutarate, 4 to 8-arm PEG-acrylate, or 4 to 8-arm PEG-propion aldehyde.

The present invention provides an extracellular microenvironment having elasiticity that can be readily controlled by selecting the concentration enhancer in biomaterial composition, whereas physical cues such as pore size and biochemical cues are consistent.

In one embodiment, biomaterial compositions to provide microenvironment having elasticity from 0.1 kpa to 2 kpa whereas average pore size is constantly 100 μm and constant biochemical cues.

Pore size of a scaffold can affect cell behavior within a scaffold and that subtle changes in pore size can have a significant effect on cell behavior.

If the pores become too large the mechanical properties of the scaffold will be compromised due to void volume and as pore size increases further, the specific surface area will eventually reduce to a level that will limit cell adhesion.

Cellular activity is influenced by specific integrin-ligand interactions between cells and surrounding ECM. Initial cell adhesion mediates all subsequent events such as proliferation, migration and differentiation within the scaffold. As a result the mean pore size within a scaffold affects cell adhesion and ensuing proliferation, migration and infiltration. Therefore maintaining a balance between the optimal pore size for cell migration and specific surface area for cell attachment is essential. (Ma Z, et al., Potential of nanofiber matrix as tissue-engineering scaffolds. Tissue Eng. 2005. 11(1-2):101-9; Karageorgiou V, Kaplan D. Porosity of 3D biomaterial scaffolds and osteogenesis. Biomaterials. (2005) 26(27):5474-91.)

As summarized in Table 2, the optimal pore size will vary with different cell types (O'Brien F J, et al., The effect of pore size on cell adhesion in collagen-GAG scaffolds. Biomaterials. 2005; 26(4):433-41). A recent study demonstrated that mesenchymal stem cells seeded on the smaller range of CG scaffolds and maintained in osteogenic culture for 3 weeks showed improved osteogenesis on the scaffolds with bigger pores (Byrne E M, et al., Gene expression by marrow stromal cells in a porous collagen-glycosaminoglycan scaffold is affected by pore size and mechanical stimulation.) Mater Sci Mater Med. 2008 November; 19(11):3455-63).

TABLE 2
Optimal pore size for cell infiltration and host tissue ingrowth

| Cell/tissue type | Pore size (μm) | | Scaffold material |
|---|---|---|---|
| Human skin fibroblasts | <160 | μm | PLA/PLG |
| Bone | 450 | μm | PMMA |
| Fibrocartilaginous tissue | 150-300 | μm | Polyurethane |
| Adult mammalian skin cells | 20-125 | μm | Collagen-GAG |
| Osteogenic cells | 100-150 | μm | Collagen-GAG |
| Smooth muscle cells | 60-150 | μm | PLA |
| Endothelial cells | <80 | μm | Silicon nitride |

The present invention also provides a synthetic microenvrionment that precisely regulate cell growth, proliferation or differenation by presenting growth factor mimetic peptide motif that interacts with integrin to induce synergistic effect on such cellular behaviors.

A growth factor is a naturally occurring polypeptide capable of regulating cell proliferation and differentiation. Growth factors are important for regulating a variety of physiological processes including tissue development, regeneration, and wound healing.

For example, fibroblast growth factors stimuate most cells to promote mitogenic and non-mitotic response to FGF. FGFs can activate cell's migration to wound healing (chemotatic), blood vessel formation (angiogenesis), regulation of nerve cell regenration (guided neuronal growth), expression in specific cells, promotion or suppression of cell survival (Ornitz and Itoh, Fibroblast growth factors, Genome Biology 2001 2(3), 3005.1-3005.12).

Today FGF family consists of 23 members including acidic and basic fibroblast growth factor, and each FGF has canofin, hexfin, and decafin motif as active domains (Li S, et al., Fibroblast growth factor-derived peptides: functional agonists of the fibroblast growth factor receptor. J Neurochem. 2008 February; 104(3):667-82., Li S, et al., Agonists of fibroblast growth factor receptor induce neurite outgrowth and survival of cerebellar granule neurons. Dev Neurobiol. 2009. 69(13):837-54., Shizhong Li, et al., Neuritogenic and Neuroprotective Properties of Peptide Agonists of the Fibroblast Growth Factor Receptor. Int J Mol Sci. 2010; 11(6): 2291-2305).

MAPTrix™ FGF mimetic has a similar bioactivity to natural or recombinant fibroblast growth factor, where the mussle adhesive protein was recombinantly functionalized with fibroblast growth factor (FGF) including acidic FGF derived peptide TGQYLAMDTDGLLYGS (SEQ ID NO: 91), WFVGLKKNG SCKRG (SEQ ID NO: 92), basic FGF derived peptide, HFKDPKRLYCK (SEQ ID NO: 93), FLPMSAKS (SEQ ID NO: 94), KTGPGQKAIL (SEQ ID NO: 95), ANRYLAMKEDGRLLAS (SEQ ID NO: 96), WYVALKRTGQYKLG (SEQ ID NO: 97), FGF-3 derived peptide SGRYLAMNKRGRLYAS (SEQ ID NO: 107), FGF-9 derived peptide SGLYLGMNEKGELYGS (SEQ ID NO: 108), FGF-10 derived peptide SNYYLAMNKKGKLYGS (SEQ ID NO: 109), FGF-17 derived peptide SEKYICMNKRGKLIGK (SEQ ID NO: 110).

The present invention provides a synthetic microenvironment to induce endothelial tube formation by mussel adhesive protein recombinatly functionalized with peptide (SEQ ID NO: 93) by presenting synergistic interaction of integrin-fibroblastic growth factor mimetic.

Similarly, a mussel adhesive protein can be recombinantly functionalized with peptides derived from a variety of growth factor proteins including TGF-α derived peptide HADLLAVVAASQ (SEQ ID NO: 111), TGF-β derived peptide KVLALYNK (SEQ ID NO: 112), EGF derived peptide CMHIESLDSYTC (SEQ ID NO: 113), NGF derived peptide PEAHWTKLQHSLDTALR (SEQ ID NO: 114), PDGF derived peptide, SVLYTAVQPNE (SEQ ID NO: 115), VEGF derived peptide KLTWQELYQLKYKGI (SEQ ID NO: 116)

A synthetic microenviornment-forming composition comprised of the 8-arm PEG-SG can be readily formed with mussel adhesive proteins functionalized with ECM derived peptides; or, a hydrogel-forming composition comprised of the 6-arm PEG-SG can be formed with mussel adhesive proteins, or a mixture of mussel adhesive proteins with 6-arm PEG-amine etc

The present invention provides a synthetic microenvironment comprised of biomaterial composition including hyaluronic acid. Hyaluronic acid (also called Hyaluronan or hyaluronate or HA) is an anionic, nonsulfated glycosaminoglycan distributed widely throughout connective, epithelial, and neural tissues. As a main component of the extracellular matrix, hyaluronic acid contributes significantly to cell proliferation and migration, and storage and diffusion of cellular growth factors, nutrients. It also play a role in intestitial mainetance (J. Necas, et al., Hyaluronic acid (hyaluronan): a review. Veterinarni Medicina, 53, 2008 (8): 397-411).

Mussel adhesive protein is a positively charged due to lysine-rich and hyaluronic acid is a negatively charged and thus it is hard to form a hydrogel because of the electrostatic interaction between MAPTrix™ and hyaluronic acid, leading to aggregate formation.

In one embodiment, a hydrogoel comprised of MAPTrix™ and hyaluronic acid can be easily made by pegylating MAPTrix™ to reduce such electrostatic interaction between amine groups in lysine residues and carobxylic acid in hyaluronic acid.

Protein pegylation is a state of art technology and has been used to enhance the delivery of protein therapeutics. A typical example of pegylation technique that was presented by Roberts can be used in the present invention. (Roberts M J, Chemistry for peptide and protein PEGylation. Adv Drug Deliv Rev. 2002. 54(4):459-76. and Bailon P, Won C Y., PEG-modified biopharmaceuticals. Expert Opin Drug Deliv. 2009. 6(1):1-16).

Hyaluronic suitable for use in this invention may be selected to have a wide range of molecular weights, generally from as low as 1,000 up to 3 millions of Daltons. Preferably, the selected hyaluronic acid has a molecular weight of 10,000 to 500,000.

Basement membrane, a specialized sheet of extracellular matrix, is composed of four main components (laminin, collagen IV, entactin and perlecan) constitues 98% of extracellular matrix proteins, and the remaining including hyaluronic acid, heparan, and collagenase constitutes 2%. (Valerie S. LeBleu et al., Structure and Function of Basement Membranes. Exp Biol Med 2007 232(9). 1121-1129).

In one embodiment, generally the weight ratio of hyaluronic acid is not limited, but the composition of a synthetic microenvrionment is similar to native extracellular matrix, for example, a preferred weight ratio of hyaluronic acid is between 0.1 wt % and 40 wt %, more preferably 0.5 wt % and 2 wt %.

The present invention provides an architecture controlled synthetic microenvironment. It is well known that scaffold architecture such as morphology affects cell binding and spreading. For example, cells binding to scaffolds with microscale architectures flatten and spread as if cultured on flat surfaces. Scaffolds with nanoscale architectures have larger surface areas to adsorb proteins, presenting many more binding sites to cell membrane receptors, significantly affecting cellular shape or activities. (M. M. Stevens and J. H. George, Exploring and engineering the cell-surface interface, Science, Vol. 310 (2005) 1135-8).

The porosity and pore architecture in terms of porosity and pore architecture play a significant role in cell survival, proliferation, and migration, and thus they are key elements to design a synthetic three dimensional microenvironment. (Annabi N. et al., Controlling the Porosity and Microarchitecture of Hydrogels for Tissue Engineering. Tissue Eng Part B Rev. 2010. 16(4):371-83). The porosity of a hydrogel depends on PEG molecular weight, concentration, acidity, gelation temperature and gelation time.

The present invention provides a crosslinkable biomaterial composition for porosity and pore architecture-controlled microenvironment by controlling MAPTrix™ concentration and the molecular weight and concentration of multi-arm PEG.

In one embodiment of the present invention, a synthetic microenvironment with its pore size having 0.1 to 1,000 μm is presented. Preferably, a synthetic microenvironment with its pore size having 0.1 to 100 μm is presented.

The present invention also provides a modulatory microenvironment by presenting matricryptic sites having one of the following formulae; MAP-ECM-X-NH₂ or MAP-ECM1-X-ECM2-Y-NH₂, wherein MAP is a recombinant mussel adhesive protein selected from FP1, FP2, FP3, FP4, FP5 FP6 or the combination thereof including FP151 fusion protein (SEQ ID NO: 15), ECM is a peptide motif derived from ECM or growth factor, X and Y are an enzyme sensitive peptide motif having the same or different enzymatic degradation rates.

The end terminal amine groups present in this formula can be utilized to crosslink with said multi-arm PEG to form a hydrogel having matricryptic sites as described in FIG. 1.

Matricryptic sites are biologically active sequences within ECM proteins that are not exposed in the soluble form of a molecule, but may be expressed following structural or conformational changes to the protein. These sequences represent a unique reserve of signaling sites in connective tissue that may be exposed and activated under a variety of conditions where ECM remodeling occurs. Mechanisms that promote matricryptic site expression include protein multimerization, proteolysis, and mechanical forces. (Davis G E, et al, Regulation of tissue injury responses by the exposure of matricryptic sites within extracellular matrix molecules. Am J Pathol. 2000; 156: 1489-1498.)

The microenvironment of cells in vivo is defined by spatiotemporal patterns of chemical and biophysical cues; and, cellular behavior is precisely regulated by theses cues within the extracellular environment that vary across time and space (Richter C, et al., Spatially controlled cell adhesion on three-dimensional substrates. Biomed Microdevices. 2010 October; 12(5):787-95.).

Therefore, introducing a dynamic aspect, i.e. the ability to modulate cell-substrate interaction with an external stimulus, opens up many further opportunities in designer surfaces for cell culture or tissue engineering applications.

Our approach is to incorporate a matricryptic sites into mussel adhesive proteins. The matricryptic site comprises at least one or more enzyme sensitive peptide incorporated into the ECM derived peptide having a formula of MAP-ECM-X-NH₂ or MAP-ECM1-X-ECM2-Y-NH₂.

A hydrogel-forming composition comprising said matricryptic site containing mussel adhesive protein can easily form matricryptic sites containing 3D microenvironments. The degradation of hydrogels can be engineered to occur, for example, via cellsecreted enzymes such as matrix metalloproteinase or collagenase. Upon hydrogel degradation, cells become exposed to ECM peptides, triggering signaling events to regulate cellular behavior.

Suitable enzyme sensitive peptide motifs are derived from collagenase, elastase, factor XIIIa, matrix metalloproteases (MMPs) or thrombin.

Preferably, an enzyme sensitive peptide fragment derived from MMPs is a GPQGIAGQ(SEQ ID NO: 65), GPQGIASQ(SEQ ID NO: 66), GPQGIFGQ(SEQ ID NO: 67, GPQGIWGQ(SEQ ID NO: 68), GPVGIAGQ(SEQ ID NO: 69), GPQGVAGQ(SEQ ID NO: 70) or GPQGRAGQ(SEQ ID NO: 71)

Preferably, an enzyme sensitive peptide fragment derived from collagenase is a LGPA (SEQ ID NO: 72) or APGL (SEQ ID NO: 73).

Preferably, an enzyme sensitive peptide fragment derived from factor XIIIa is a NQEQVSP (SEQ ID NO: 74).

Preferably, an enzyme sensitive peptide fragment derived from elastase is a AAAAAAAA (SEQ ID NO: 75).

Preferably, an enzyme sensitive peptide fragment derived from plasmin is YKNR(SEQ ID NO: 76), NNRDNT(SEQ ID NO: 77), YNRVSED(SEQ ID NO: 78), LIKMKP(SEQ ID NO: 79), or VRN(SEQ ID NO: 80).

Preferably, an enzyme sensitive peptide fragment derived from thrombin is GLVPRG (SEQ ID NO: 81).

In one embodiment of the present invention, there is an enzyme digestible composition of a modulatory 3D microenviornment which is comprised of a mussel adhesive protein that is recombinatly functionalized with matrix metalloprotease (MMP) sensitive peptide motifs which are incorporated into laminin derived peptide motifs having the formula AKPSYPPTYKAKPSYPPTYK-IKVAV-GPQGIAGQ (SEQ ID NO: 82), AKPSYPPTYKAKPSYPPTYK-GFPGER-GPQGIAGQ (SEQ ID NO: 83), AKPSYPPTYKAKPSYPPTYK-GRGDSP-GPQGIAGQ (SEQ ID NO: 84), or AKPSYPPTYKAKPSYPPTYK-GRGDSP-IKVAV-GPQGIAGQ(SEQ ID NO: 85)

In one embodiment of the present invention, there is an enzyme digestible composition of a modulatory 3D microenviornment which is comprised of a mussel adhesive protein FP-1 (SEQ ID NO: 3) or FP-151 (SEQ ID NO: 15) that is recombinatly functionalized with matrix metalloprotease (MMP) sensitive peptide motifs which are incorporated into collagen type I and laminin derived peptide motifs having the formula AKPSYPPTYKAKPSYPPTYK-IKVAV-GPQGIAGQ-GFPGER-GPQGIWGQ (SEQ ID NO: 86) or AKPSYPPTYKAKPSYPPTYK-IKVAV-GPQGIAGQ-GRGDSP-GPQGIWGQ (SEQ ID NO: 87).

In another embodiment of the present invention, there is an enzyme digestible composition of a modulatory 3D microenviornment which is comprised of a mussel adhesive protein FP-1 (SEQ ID NO: 3) or FP-151 (SEQ ID NO: 15) that is recombinatly functionalized with matrix metalloprotease (MMP) sensitive peptide motifs which are incorporated into collagen type I and laminin derived peptide motifs having the formula AKPSYPPTYKAKPSYPPTYK-IKVAV-GPQGIAGQ-GFPGER-GPQGIWGQ(SEQ ID NO: 86) or AKPSYPPTYKAKPSYPPTYK-IKVAV-GPQGIAGQ-GRGDSP-GPQGIWGQ(SEQ ID NO: 87) incorporated between FP-1 (SEQ ID NO: 3) and FP-5 (SEQ ID NO: 15).

The present invention can be used in high throughput screening (HTS) to identify a combination of peptide motifs to screen and design an optimal synthetic microenvironment that induces combinatorial signaling to regulate specific cellular behavior. The screening for an appropriate differentiation environment of stem cells is an especially urgent issue in the fields of regenerative medicine and drug discovery.

The MAPTrix™ hydrogel can be in the form of a hydrogel array for high throughput screening. A “hydrogel array” is a combination of two or more microlocations. Preferably an array is comprised of microlocations in addressable rows and columns. The thickness and dimensions of the MAPTrix™ hydrogel and/or hydrogel arrays produced according to the invention can vary, dependent upon the particular needs of the end-user.

The invention provides for a device of MAPTrix™ hydrogel array comprising:

(a) obtaining a crosslinkable MAPTrix™ composition;

(b) placing a crosslinkable MAPTrix™ composition on a solid support in a pattern; and

(c) crosslinking the MAPTrix™ composition to obtain the MAPTrix™ hydrogel array.

In one embodiment of this invention, a MAPTrix™ hydrogel array is provided. The array is a 96-well, microtiter plate consisting of 12×8-well removable strips. Each well within a strip (7 wells total) is pre-coated with a different MAPTrix™ ECM composition to generate a different 3D microenvironment (see FIG. 1) along with one reconstituted basement membrane-coated well as a positive control. Cells of interest can be seeded onto each well, whereby cells are cultured in a different 3D microenvironment. A synthetic 3D microenvironment that induces a desirable cellular behavior can be identified and designed from the assay utilizing this MAPTrix™ hydrogel array.

### BRIEF DESCRIPTION OF DRAWINGS

These and other features and advantages of the invention are evident from the following embodiments when read in conjunction with the accompanying drawings in which;

FIG. 1 shows the schematic representation of a modulatory 3D microenvironment. 1A) a hydrogel formed from MAP-ECM-X-NH₂, 1B) a hydrogel formed from mixture of MAP-ECM-X-NH₂ and MAP-ECM-Y-NH₂ wherein X and Y are enzyme sensitive motif having different enzyme cleavage rates, 1C) a hydrogel formed from MAP-ECM-X-ECM-Y-NH₂.

FIG. 2 represents modulus of each synthetic matrix having the same pore size of 100 μm.

FIGS. 3a and 3b represent scanning electromicrographs of the effect of MAPTrix™ Fibronectin concentration on pore size. FIG. 3a: a hydrogel from MAPTrix™ concentration 15 mg/ml, and FIG. 3b: a hydrogel from MAPTrix™ concentration 20 mg/ml.

FIGS. 4a to 4d represent scanning electron micrographs of each MAPTrix™ HyGel having the same pore size but having different modulus. FIG. 4a: 892 Pa, FIG. 4b: 576 Pa, FIG. 4c: 510 Pa, FIG. 4d: 621 Pa.

FIG. 5 represents MAP containing enzyme sensitive motif was digested by type IV bacterial collagenase. MAP is a recombinant mussel adhesive protein and E-MAP contains MMP sensitive motif GPQGIAGQ sensitive to a variety of collagenase including MMP-1, MMP-2, MMP-3, and MMP-9.

FIGS. 6a and 6b represent a microenvironment array to screen optimal extracellular microenvironment for keratinocyte. Combinations of adhesion and signal molecules were coated onto 96 well surface. ECM compositions with varying weight ratio of collagen-derived integrin binding motif to heparin and growth factor receptor binding motif (FIG. 6a), and fibronectin derived integrin binding motif to heparin and growth factor receptor binding motif (FIG. 6b).

FIGS. 7a and 7b represent a layout of extracellular microenvironment array for screening of an optimal combinatorial integrin-mediated signaling which can induce endothelial tube formation

FIGS. 8a and 8b represent the MAPTrix™ Fibronectin solution mixed with hyaluronic acid solution to mimic a native extracellular matrix. Pegylated MAPTrix™ solution was transparent whereas the mixture of MAPTrix™ ECM and hyaluronic acid was not transparent A) MAPTrix™ Fibronectin solution before mixing with hyaluronic acid, B) MAPTrix™ Fibroenctin solution mixed with hyaluronic acid. The left vial contained pegylated MAPTrix™ Fibronectin and the right vial contained MAPTrix™ Fibronectin

FIGS. 9a and 9b represent cell adhesion profiles of HaCaT cultured on MAPTrix™ microenvironment arrays. Cell counts were normalized against average cell counts on MAPTrix™ having no any bioactive peptide. Each bar represents the mean value of three wells. FIG. 9a: Effect of combinatorial signaling of integrin α1β1/α2β1 and heparin derived from collagen or vitronectin on HaCaT adhesion and growth. FIG. 9b: Effect of combinatorial signaling of α1β1/α2β1 and basic FGF and EGF mimetics on HaCaT adhesion and growth.

FIG. 10 shows the effect of a single and combinatorial presentation of ECM peptide motifs on tube formation. MAPTrix™ ECM containing the combination of αvβ3-α2β1 integrin binding motifs provided the best favorable environment for endothelial tube formation. MAPTrix™ ECM containing the combination of αvβ3-α5β1 integrin binding motifs provided a normal environment for endothelial tube formation.

FIG. 11a to 11c show temporal course of endothelial tube formation. FIG. 11a: Single presentation of angiogenic integrin binding peptide. FIG. 11b: Combinatorial presentation of angiogenic integrin and syndecan binding peptides. FIG. 11c: Combinatorial presentation of two different angiogenic integrins.

FIGS. 12a and 12b show the endothelial tube formation cultured on reconstituted basement membrane, GelTrex (Invitrogen).

FIG. 13 shows the temporal course of endothelial tube formation on the integrin binding motifs that provided for a favorable environment.

FIG. 14 shows the effect of physical properties on endothelial tube formation in serum free conditions. The porer size was controlled by the type of PEG-SG type.

FIGS. 15a to 15c show the effect of physical cues on endothelial tube formation. Hydrogel having two different pore architures and pore size was created by chaning gelation temperature. A gel that underwent thermal annealing had more compact structured 3D micreonvironment. Macroporous structure with good porosity supported endothelial tube formation when αvβ3-α2β1 (50/50 to 75/25 in weight) whereas the gel with fibrous structure lacking porosity did not induce the tube formation in the same signaling environment.

FIG. 16 shows the concentration effect of MAPTrix™ FGF as FGF mimetic on FGFR1 phosphorylation. MAPTrix™ FGF has a similar bioactivity to recombinant bFGF at 50 to 100 higer concentration.

FIGS. 17a and 17b show the comparison of MAPTrix™ FGF with recombinant bFGF in endothelial tube formation of HUVEC cultured on MAPTrix™ HyGel representing fibronectin derived REDV (SEQ ID NO: 61).

FIG. 18 shows cell morphology of human dermal fibroblast cultured on MAPTrix™ HyGel having different concentration of enzyme sensitive motifs. When the weight ratio of Dynamic MAPTrix™ to MAPTrix™ was 75:25 to 50:50, the dermal fibroblast formed a tube-like shape as in reconstituted basement membrane GelTrex.

FIG. 19 shows effect of serum on the morphology of fibroblast cultured on Dynamic MAPTrix™ HyGel.

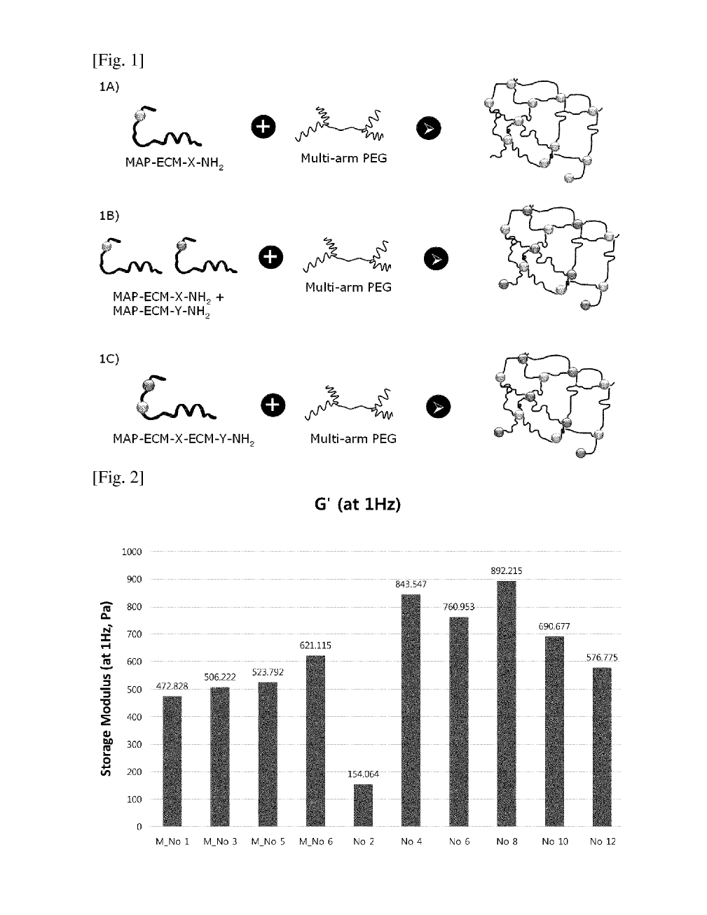

### BEST MODE FOR CARRYING OUT THE INVENTION

The following EXAMPLES are provided to demonstrate preferred embodiments of the present invention and the invention is not intended to be limited in scope by the specific embodiments described herein, which are intended for the purposes of exemplification only. Functionally equivalent products, compositions and methods are clearly within the scope of the invention, as described herein.

### Example 1

### Creation of MAPTrix™ HyGel which Provides a Biochemically Defined Microenvironment

MAPTrix™ HyGel gel solution was prepared as follows: A synthetic 3D microenvironment to mimic naturally occurring extracellular matrix that induce a combinatorial signaling was created as summarized in Table 2. The final concentration of each ECM composition at 20 mg/ml was prepared in PBS buffer solution (pH 7.4).

The ECM composition was composed of αvβ3 binding peptide motif derived from laminin α1 chain based combination to induce combinatorial signaling of αvβ3-α2β1, αvβ3-α3β1, αvβ3-α5β1, αvβ3-syndecan. 20 mg/ml. The MAPTrix™ ECM solution was mixed with 20 mg/ml 4 ARM and 8 ARM PEG-SG, (1:1 (v/v)) dissolved in PBS buffer (pH 7.4). The prepared MAPTrixHyGel™ solutions were added to a 48-well plate (BD Biosciences) and allowed to gel at 37° C. for 2 h.

The prepared MAPTrixHyGel™ solutions were added to a 48-well plate (BD Biosciences) and allowed to gel at 37° C. for 2 h.

As presented in FIGS. 7a and 7b, a layout of 48 well plate was coated with laminin α1 chain derived αvβ3 binding peptide motif IKVAV (SEQ ID NO: 37) based a variety of extracellular matrix (ECM) compositions and the concentration gradient of each ECM composition to elucidate the effect of each ECM composition and its concentration gradients on cellular behavior at the same time.

ECM composition to induce combinatorial signaling of αvβ3-α2β1, αvβ3-α3β1, αvβ3-α5β1, αvβ3-syndecan was created along with each row of microwell and a concentration gradient of ECM compositions is created along with the column of microwell to quantify the effect of each integrin binding motif on cellular behavior.

Typically, a biochemical microenviornment was created to mimic naturally occurring endothelial extracellular matrix by presenting a variety of ECM derived peptide in combination including collagen type I derived α1β1 or α2β1 binding peptide motif (SEQ ID NO: 22), DGEA (SEQ ID NO: 23), laminin α1 derived NRWHSIYITRFG (SEQ ID NO: 38), laminin β1 derived peptide motif YIGSR (SEQ ID NO: 44), fibronectin domain III derived α4β1 binding peptide motif REDV (SEQ ID NO: 61), fibronectin domain III derived α5β1 binding peptide motif GRGDSP (SEQ ID NO: 52), syndecan binding motif SPPRRARVT (SEQ ID NO: 56) derived from fibronectin and RKRLQVQLSIRT (SEQ ID NO: 40) derived from laminin α1 chain.

TABLE 3
Composition of MAPTrix ™ ECM (in weight percentage)

| αvβ3/ | αvβ3/ | αvβ3/ | αvβ3/ | αvβ3/ | αvβ3/ |
|---|---|---|---|---|---|
| GFPGER | YIGSR | REDV | GRGDSP | SPPRRARVT | RKRLQVQLSIRT |
| 100/0 | 100/0 | 100/0 | 100/0 | 100/0 | 100/0 |
| 75/25 | 75/25 | 75/25 | 75/25 | 75/25 | 75/25 |
| 50/50 | 50/50 | 50/50 | 50/50 | 50/50 | 50/50 |
| 25/75 | 25/75 | 25/75 | 25/75 | 25/75 | 25/75 |
| 0/100 | 0/100 | 0/100 | 0/100 | 0/100 | 0/100 |

### Example 2

### Creation of MAPTrix™ HyGel which Provides a Mechanically Defined Microenvironment

MAPTrix™ HyGel samples were prepared in the 6 well plate with different weight ration of multi-arm PEG-SG and poly(vinly alcohol) as an enhancer. The compositions of each MAPTrix™ HyGel sample were described in Table 2. MAPTrix™ was dissolved to a final concentration of 20 mg/ml and 40 mg/ml in PBS buffer solution (pH 7.4), respectively, which was mixed with 50 mg/ml and 100 mg/ml poly(vinyl alcohol)(PVA, 100,000 daltons) dissolved in PBS buffer solution, respectively. The mixed MAPTrix™/PVA solution was further mixed with crosslinking solution of 20 mg/ml 4 ARM and/or 8 ARM PEG-SG and allowed to form a matrix at 25° C. for 1 hr.

TABLE 4
Composition of MAPTrix ™ HyGel for mechanically defined microenvironment

| Code | MAPTrix | | PEG-SG | | PVA enhancer | |
|---|---|---|---|---|---|---|
| #2 | 4 | wt % (w/v), 23 kda | 4 | wt % (w/v), 8-arm | 0 | |
| #4 | 4 | wt % (w/v), 23 kda | 4 | wt % (w/v), 8-arm | 5 | wt % (w/v) |
| #6 | 4 | wt % (w/v), 23 kda | 4 | wt % (w/v), 8-arm | 10 | wt % (w/v) |
| #8 | 4 | wt % (w/v), 37 kda | 4 | wt % (w/v), 8-arm | 10 | wt % (w/v) |
| #10 | 4 | wt % (w/v), 37 kda | 4 | wt % (w/v), 8-arm | 5 | wt % (w/v) |
| #12 | 4 | wt % (w/v), 37 kda | 4 | wt % (w/v), 8-arm | 0 | |
| M1 | 4 | wt % (w/v), 23 kda | 4 | wt % (w/v), 4/8-arm | 0 | |
| M3 | 4 | wt % (w/v), 23 kda | 4 | wt % (w/v), 4/8-arm | 5 | wt % (w/v) |
| M5 | 4 | wt % (w/v), 37 kda | 4 | wt % (w/v), 4/8-arm | 0 | |
| M6 | 4 | wt % (w/v), 37 kda | 4 | wt % (w/v), 4/8-arm | 5 | wt % (w/v) |

Scanning electron microscopy was used to determine the morphology of the freeze-dried samples. All samples were cooled with liquid nitrogen and fractured immediately. A Hitachi S-4800 scanning electron microscope (S-4800, Hitachi, Tokyo, Japan) was used after coating the samples with platinum using an ion sputter (E-1030, Hitachi, Tokyo, Japan).

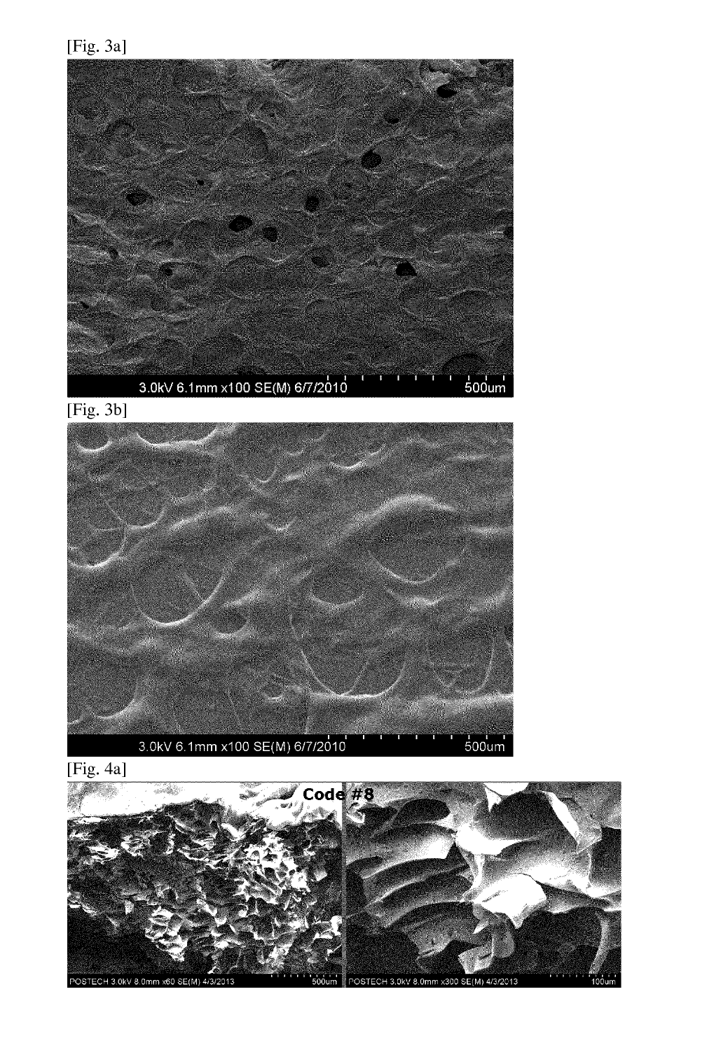

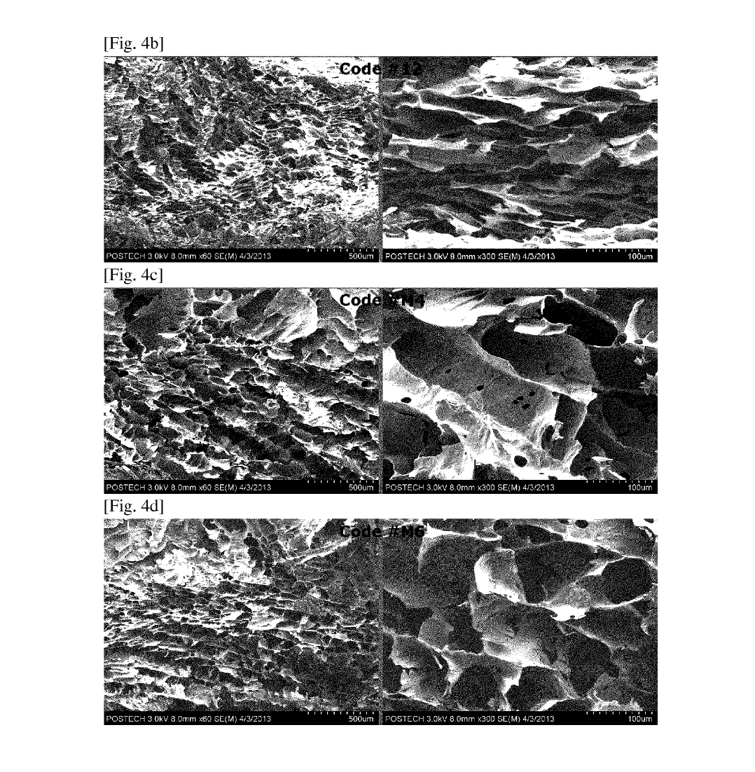

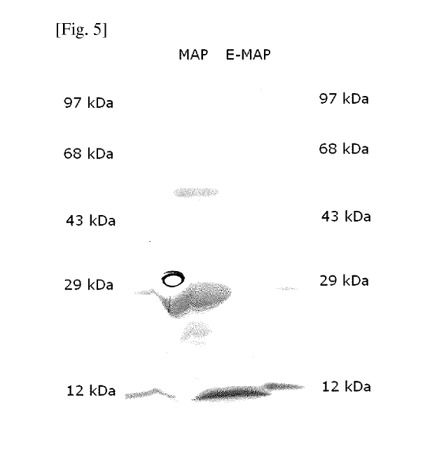

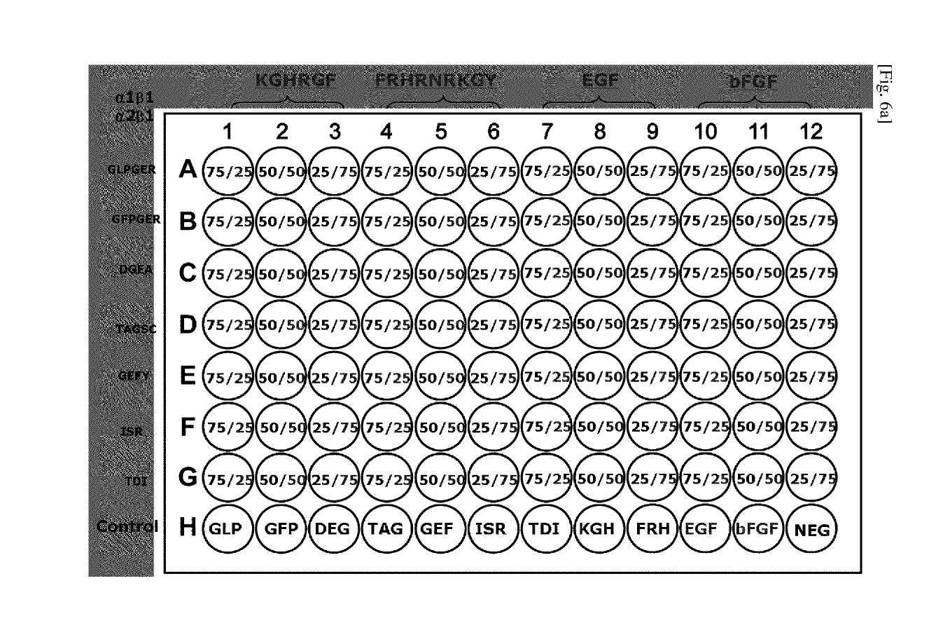

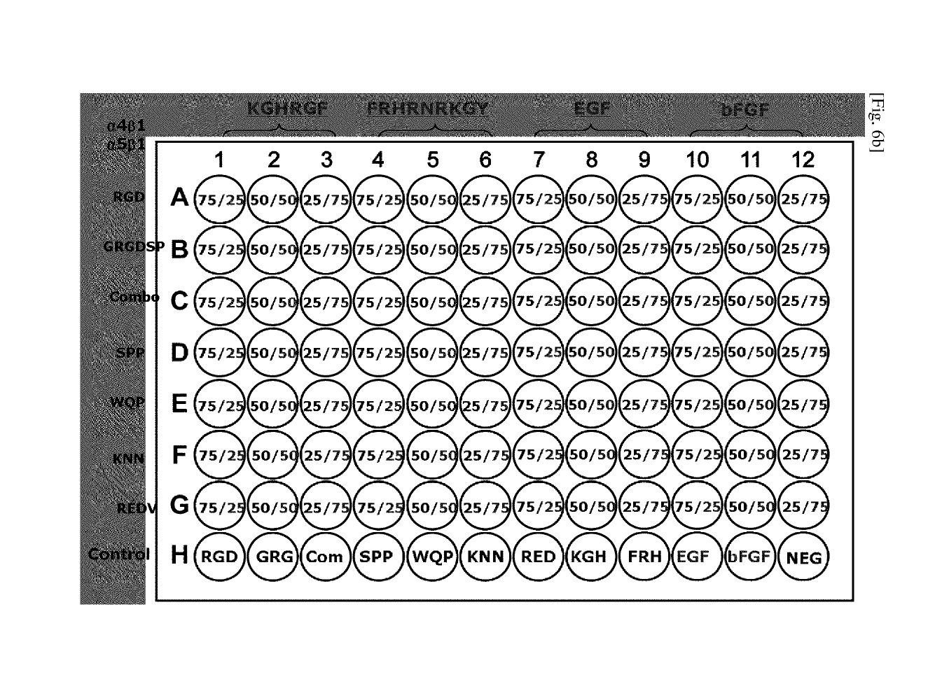

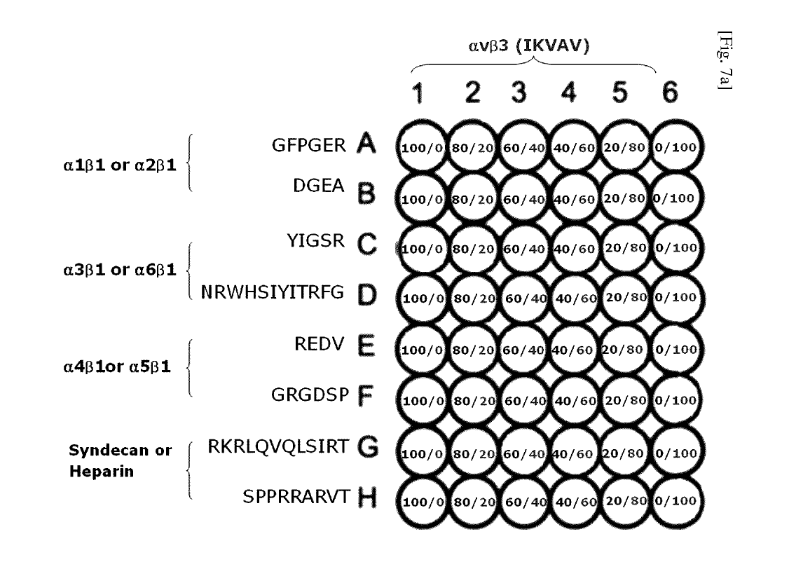

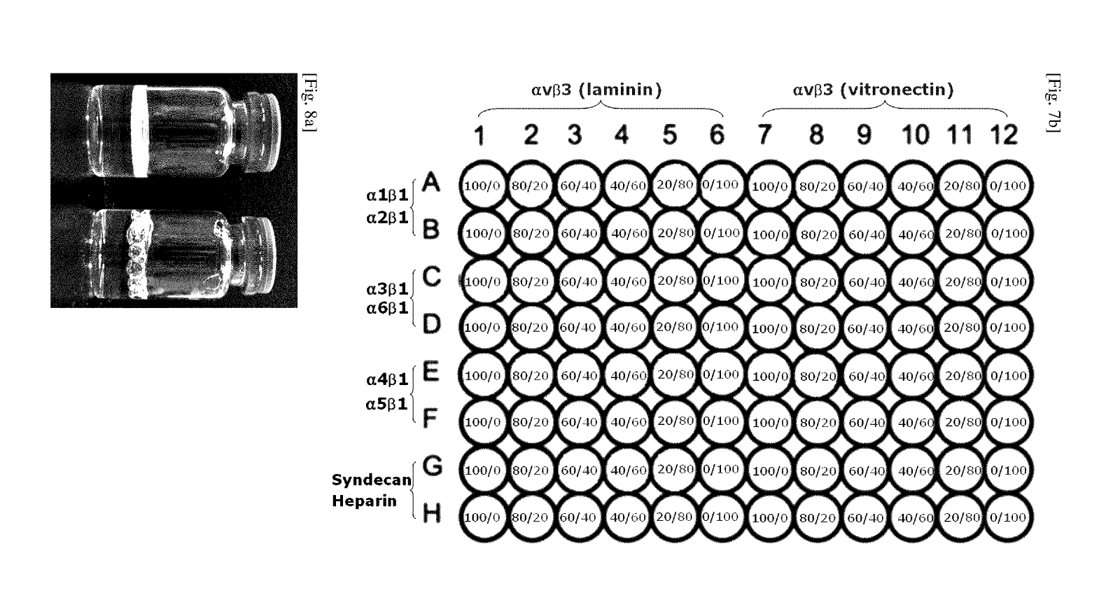

Even though each sample had different porous structure and morphology, the pore diameter of each sample was the same regardless of the concentration of MAPTrix™ and poly(vinyl alcohol) as seen in FIGS. 4a to 4d. The average pore size of all samples was about 100 μm while each sample showed different elasticity as presented in FIG. 2.

For rheology studies, the gels were prepared in the 12-well plate and swollen in the 6-well plate. Cut to a size of ˜1.2 cm in diameter, the sample was loaded onto the lower plate of the rheometer (1.3 cm in diameter), the upper fixture was lowered, and a humidity chamber was placed around the sample to prevent dehydration during data collection. The data of storage modulus (G′) and loss modulus (G″) were collected in a constant strain mode (5%) over the frequency range from 0.1 to 10 Hz.

Generally the addition of PVA to the geling solution increased the elasticity of resultant hydrogel as summarized in Table 3. However, the effect of PVA addition on increase in elasticity was significant in low molecular mussel adhesive protein (22 kda protein).

When mixture of 4-arm PEG/8-arm PEG (weight ratio=50/50) as crosslinking agent were used, the effect of MAPTrix™ molecular weight on elasticity was less significant.

During the crosslinking reaction between multi-arm PEG-SG and MAPTrix™, but the resultantly forming MAPTrix™-multi-arm PEG chains and PVA chains were interwinded to form an interpenetrating network (IPN). The presence of PVA can improve the mechanical strength as PVA based IPN hydrogel showed enhanced mechanical properties (Seon Jeong Kim, et al., Properties of interpenetrating polymer network hydrogels composed of poly(vinyl alcohol) and poly(N-isopropylacrylamide). 2003. Journal of Applied Polymer Science 89 (8). 2041-2045

Depending on the concentration of MAPTrix™, PVA, and multi-arm PEG, MAPTrix™ HyGel's elasticity ranged from 0.15 to 0.9 kPa. From EXAMPLE 2, it is evident that MAPTrix™ HyGel having 0.1 to 2 kPa can be easily prepared by adjusting the concentration and molecular weight of MAPTrix™, PVA, and multi-arm PEG-SG, whereas the pore size of each remains relatively constant.

TABLE 5
Physcial and Mechanical properties of MAPTrix ™ HyGel
based microenvironment

| | Average Pore | Storage Modulus |
|---|---|---|
| Code | Size (μm) | (Pa) |
| #2 | 100 | 156 |
| #4 | 100 | 843 |
| #6 | 100 | 760 |
| #8 | 100 | 892 |
| #10 | 100 | 690 |
| #12 | 100 | 576 |
| M1 | 100 | 472 |
| M3 | 100 | 506 |
| M5 | 100 | 523 |
| M6 | 100 | 621 |

### Example 3

### Creation of MAPTrix™ HyGel which is Enzyme-Digestible

Laminin derived peptide IKVAV coupled to a MMP sensitive motif GPQGIAGQ sequence was added to mussel adhesive protein (FP1-FP-5-Enzyme Sensitive motif-FP1) using polymerase chain reaction (PCR). The fusion protein of mussel adhesive protein and MMP sensitive IKVAV was named as Dynamic MAPTrix™ Laminin.

5 mg of Dynamic MAPTrix™ Laminin was dissolved in 10 ml PBS (1X) and type IV bacterial collagenase was added to the Dynamic MAPTrix™ Laminin solution. The ratio of MAPTrix™ Laminin:Collagenase was 25:1. After two hour incubation at 37° C., the digestion was monitored by SDS-PAGE and the apparent molecular weight of various fractions from disgested Dynamic MAPTrix™ Laminin on a SDS-PAGE gel was 29, 18, and 12 kDa. The Dynamic MAPTrix™ Laminin has 24 kDa and FP1-FP5 has 16 kDa. The apparent molecular weight of each fraction on a SDS-PAGE gel was greater than the predicted molecular mass (for example, ˜29 kDa compared with 24 kDa) due to the high pI value (9.89) of mussel adhesive protein. From the molecular weight of the fraction, we concluded that Dynamic MAPTrix™ Laminin was enzyme digestible.

### Example 4

### Preparation of Extracellullar Microenvironment Array

Microenvironment array was fabricated by covalently immobilize MAPTrix™ ECM, in combination or alone, onto the surface of 96 well plate. Series of solution of MAPTrix™ ECM (0.2 mg/ml) in 10 mM sodium acetate buffer (pH 6.5) were prepared.

100 μl of EDC (10 mM) and NHS (10 mM) solution in 10 mM sodium acetate buffer (pH 6.5) was added to each well of 96-well plate to activate COOH group and incubate for 1 hour at room temperature. After the activation, wash the plates with the cold buffer solution to completely remove the EDC/NHS reagents. 100 μl of the MAPTrix™ ECM solution to the activated 96 well plate and incubate at room temperature for 4 hours.

Extensively rinse the MAPTrix™ coated well plate with buffer solution or pure water to remove unconjugated MAPTrix™ ECM.

The concentration gradients of integrin binding motif to modulatory receptor binding motif (wt./wt.) were: 100/0, 75/25, 50/50, and 25/75, thereby creating signaling gradients via combination of integrin and modulatory receptor as represented in Figure.

### Example 5

### Cell Adhesion/Growth Assay

For keratinocyte cell adhesion and growth assays in serum free conditions, HaCaT cells were grown on the microenvironment array in Dulbecco's modified Eagle medium (DMEM, Gibco, Gaithersburg, Md.) for 24 hours. After one day, 100 μl DMEM was added to each well to wash off any non-adherent cells four times, and the add 10 μl of MTT substrate to each well and continued incubation for additional 2 hours at 37° C. MTT-treated cells were lysed and absorbance at 570 nm was measured on a spectrophotometer. Cell counts were normalized against average cell counts on MAPTrix™ having no any bioactive peptide. Each bar represents the mean value of three wells.

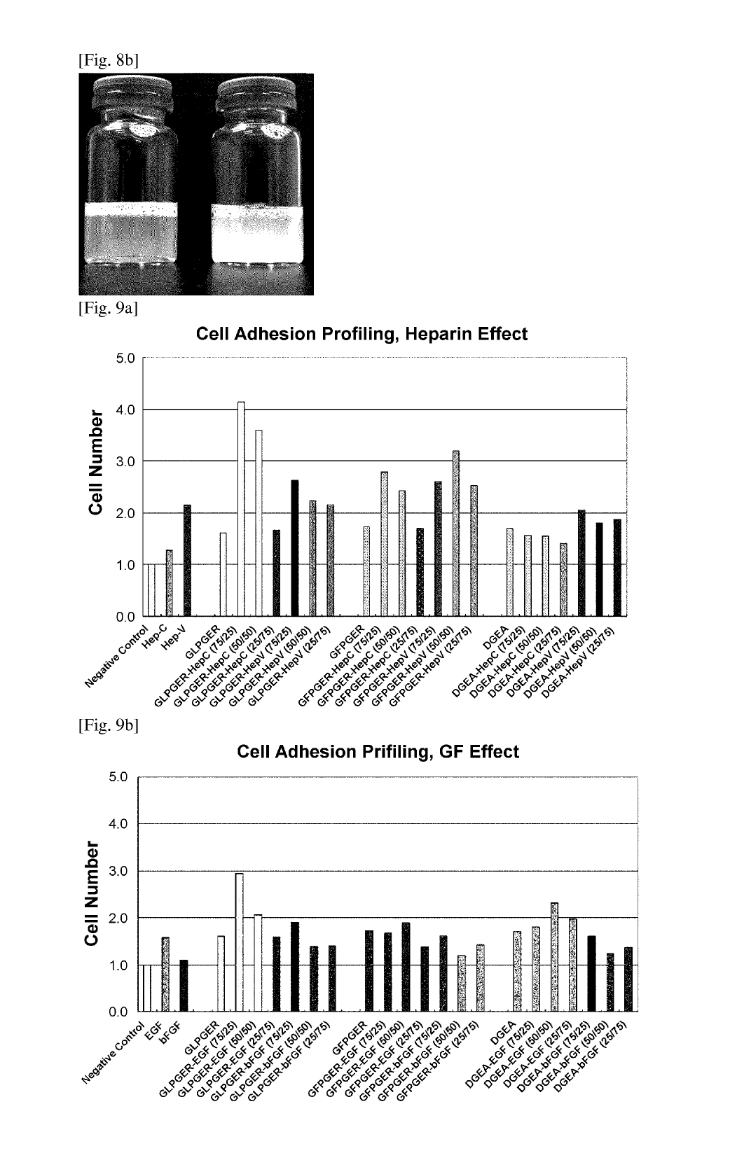

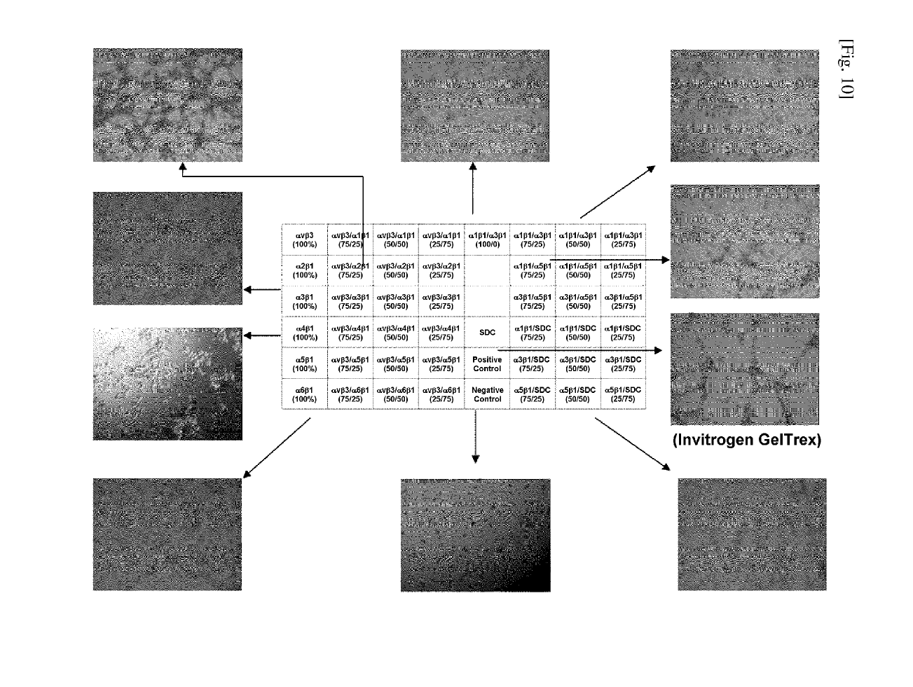

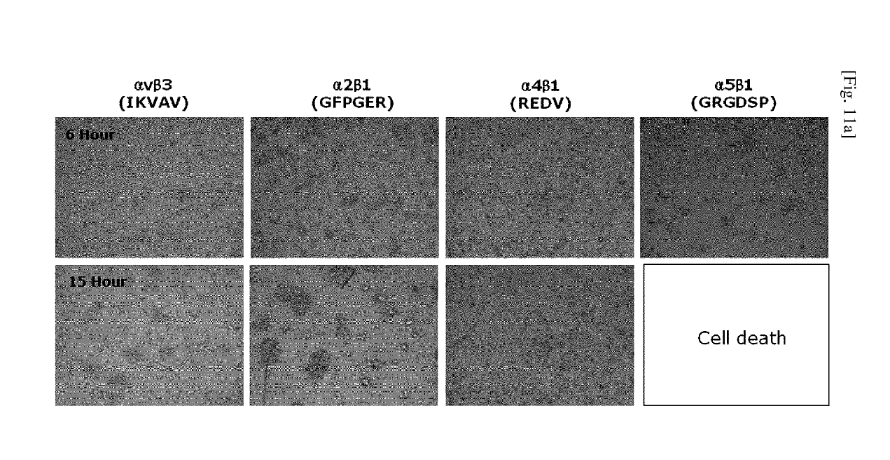

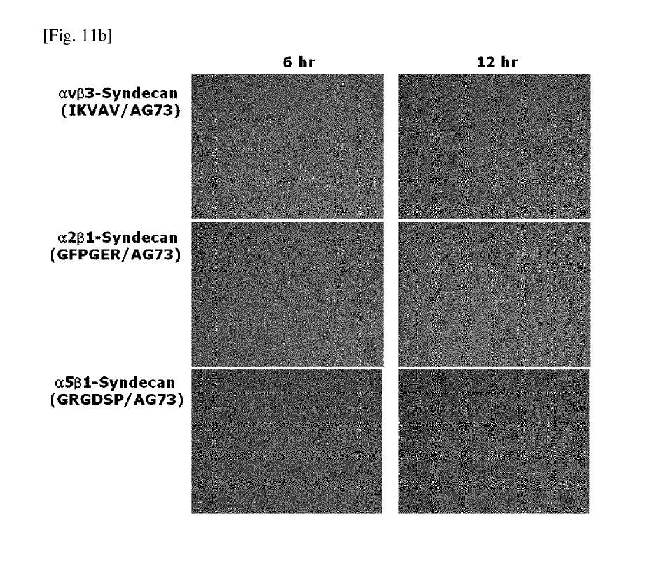

Cell adhesion/growth profiling was presented in FIGS. 9a and 9b.

MAPTrix™ without bioactive peptide was used as Negative Control. Generally combination of collagen-heparin induced more synergistic effect on cell adhesion and growth than the combination of collagen-growth factor as seen in FIGS. 9a and 9b, combinatorial signaling from collagen-derived peptide GLPGER (SEQ ID NO: 20) and heparin or growth factor mimetics offered the most favorable environment for HaCaT adhesion and growth.

Using this profiling, we have identified combinations of molecular signals that induce synergistic effect on keratinocyte growth.

The 195 signaling combinations that we analyzed could be grouped into three main groups based on their characteristic effects: (1) combinations that synergistically promoted cell adhesion and growth, (2) combinations that mildly promoted cell adhesion and growth, and (3) combinations that did not promoted cell adhesion and growth. Analysis of responses to pairs of individual signals can reveal a complex spectrum of responses to contrasting signals, which may have important implications for cell fate specification in a complex signaling microenvironments, which should be elucidated for cell therapy or tissue regeneration applications.

### Example 6

### Tube Formation Assay

Endothelial growth media (M199 media), supplemented with 10% fetal bovine serum (FBS) and endothelial cell growth supplement (ECGS, 30 μg/ml; BD Biosciences), was used to seed HUVEC cells.

HUVEC cells were washed in serum-free M199 medium by centrifuging at 400 g for 1 min, and the washed HUVECs were resuspended in serum-free M199 medium and seeded onto each MAPTrix™ hydrogel prepared from Example 1 at a density of 5×10⁴ cells/well with 100 ng/ml VEGF and incubated at 37° C. for 24 hours. The morphology of HUVECs was monitored and photographed with a phase contrast microscope at regular intervals (every 6 hours).

TABLE 6
Composition of MAPTrix ™ ECM (in weight percentage)

| αvβ3/ | αvβ3/ | αvβ3/ | αvβ3/ | αvβ3/ | αvβ3/ |
|---|---|---|---|---|---|
| GFPGER | YIGSR | REDV | GRGDSP | SPPRRARVT | RKRLQVQLSIRT |
| 100/0 | 100/0 | 100/0 | 100/0 | 100/0 | 100/0 |
| 75/25 | 75/25 | 75/25 | 75/25 | 75/25 | 75/25 |
| 50/50 | 50/50 | 50/50 | 50/50 | 50/50 | 50/50 |
| 25/75 | 25/75 | 25/75 | 25/75 | 25/75 | 25/75 |
| 0/100 | 0/100 | 0/100 | 0/100 | 0/100 | 0/100 |

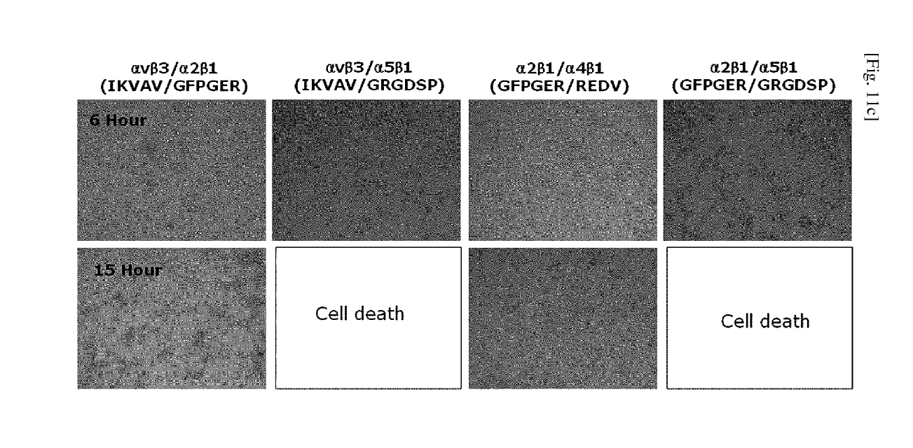

The effect of each microenvironment on the morphology of HUVEC cells was quite different as seen in the FIG. 11a (microenvironment presenting single interin-binding motif), and FIG. 11b (microenvironment presenting combinatorial interin-binding motifs).

MAPTrix™ composition presenting αvβ3 or α5β1 integrin binding motif provided a favorable environment for endothelial tube formation while MAPTrix™ composition presenting α2β1 and α4β1 integrin binding motif provided a less favorable environment for endothelial tube formation. (FIG. 11a).

MAPTrix™ composition presenting a combination of αvβ3-α2β1 integrin binding motifs provided the best favorable environment for endothelial tube formation. However, MAPTrix™ composition presenting a combination of αvβ3-α5β1 integrin binding motifs provided a normal environment for endothelial tube formation.

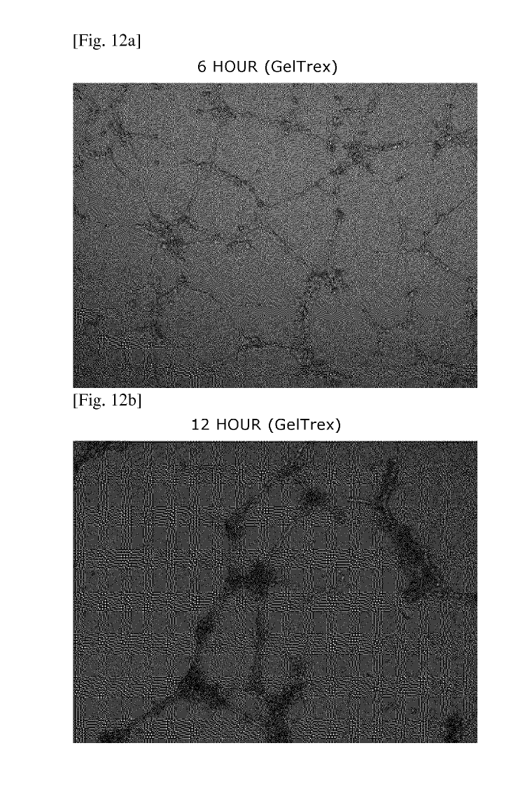

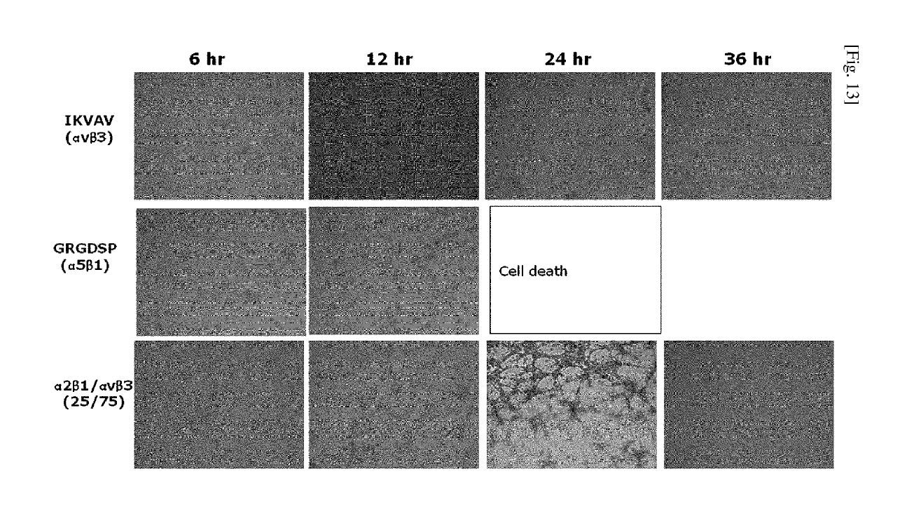

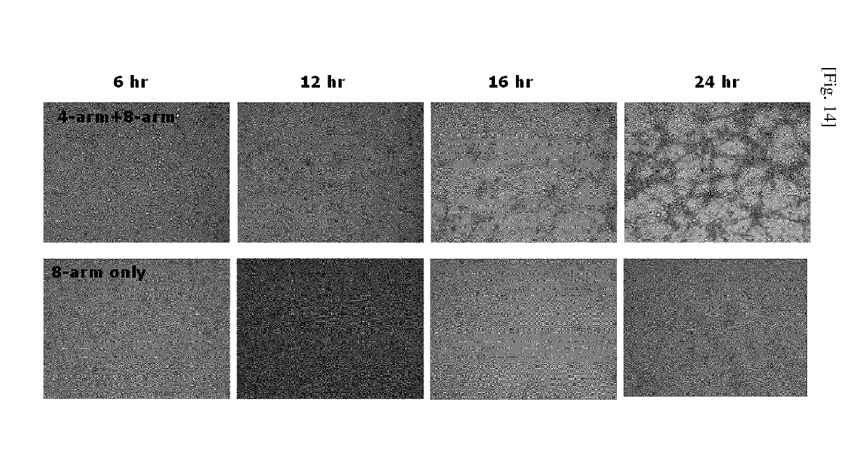

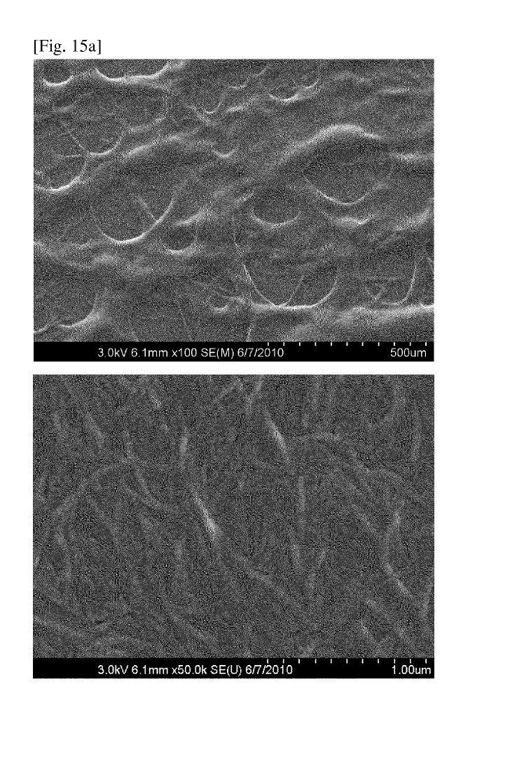

As demonstrated in FIGS. 12a and 12b and FIGS. 13a and 13b, MAPTrix™ composition presenting a combination of αvβ3-α2β1 integrin binding motifs appeared to be a similar microenvironment to a natural endothelial basement membrane, based on a morphology analysis.

### Example 7

### Effect of Physical Cues of MAPTrix™ HyGel on Endothelial Tube Formation

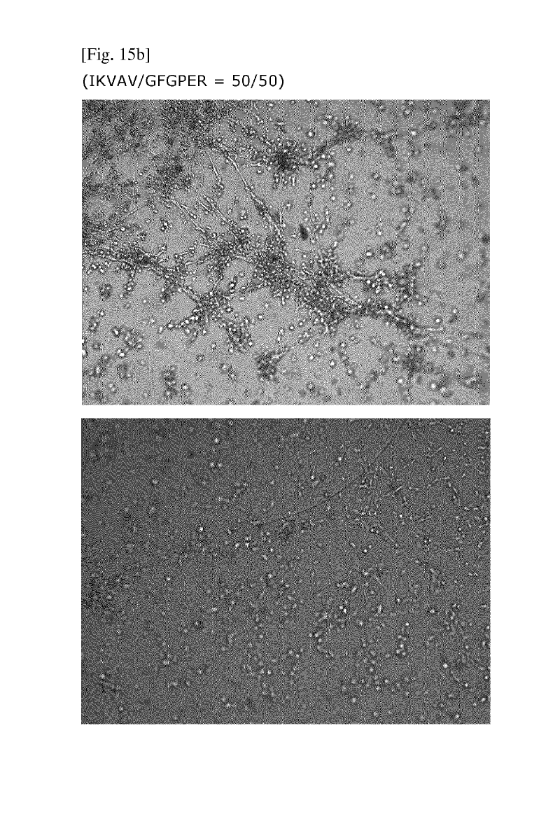

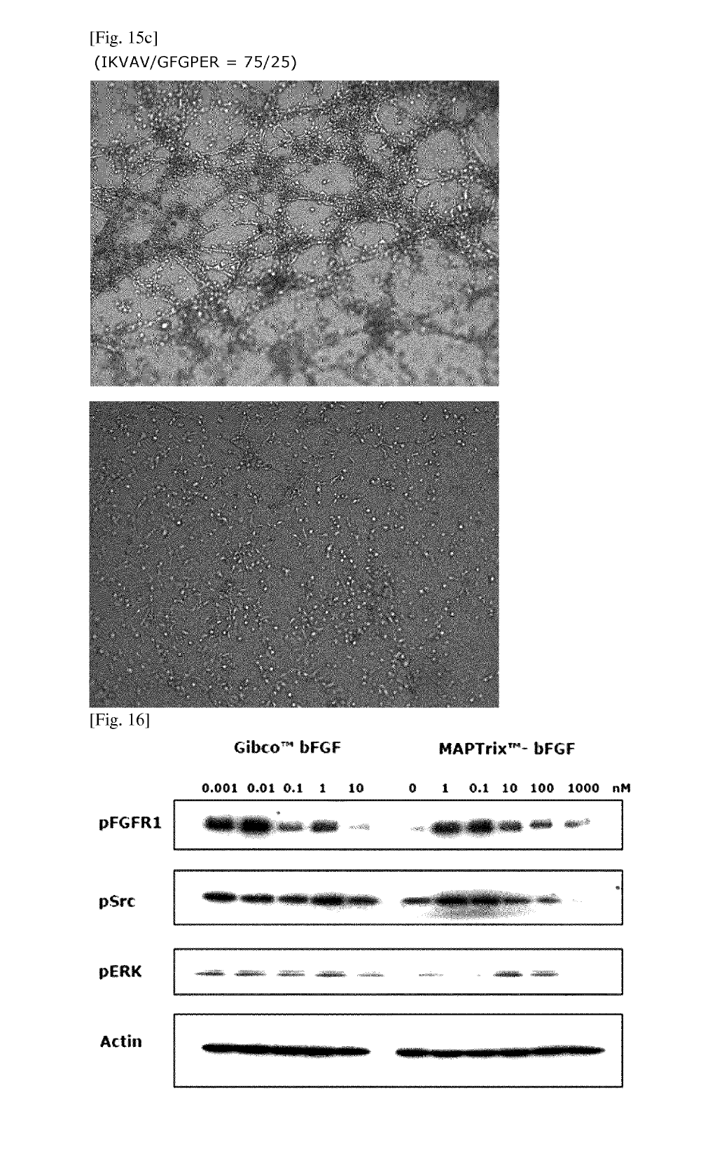

When MAPTrix™ HyGel underwent annealing, its surface morphology and elasticity was quite different from the original MAPTrix™ HyGel as seen in FIGS. 15a.

We investigated the effect of surface morphology and pore size on endothelial tube formation.

As demonstrated in FIGS. 15b and 15c, soft matrix with porous morphology induced endothelial tube formation but a hard matrix with fibrous morphology did not support endothelial tube formation when both matrices presented the same biochemical cues to HUVEC.

### Example 8

### Functional Assay of MAPTrix™ GF

Dermal fibroblast (HS27) cells were seeded in serum-free media for two days, and the serum-free media was replaced and maintained for additional one day. Cells were then treated with MAPTrix™ FGF for 5 min followed by subjecting to cell lysates to immunoblotting with antibodies to pFGFR1 and pERK. Phosphorylation levels of FGFR1 and ERK were assessed by the immunoblotting.

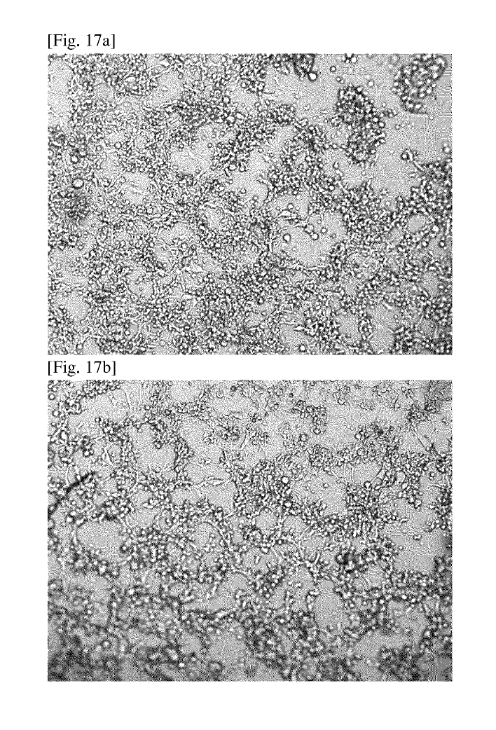

FIG. 16 indicated MAPTrix™ FGF could activate FGFR1 at high concentrations. Similar tests were conducted in HUVEC cells with the same procedure, and MAPTrix™ FGF displayed similar bioactivity to natural FGF at about 50 to 100 times higher concentration of MAPTrix™ FGF.

We prepared MAPTrix™ HyGel presenting fibronectin derived peptide, REDV (SEQ ID NO:) that did not support endothelial tube formation but promoted cell adhesion. HUVECs were seeded in serum free media for two days, and the serum-free media was replaced and maintained for additional one day. Cells were treated with MAPTrix™ FGF and recombinant bFGF and maintained for one day.

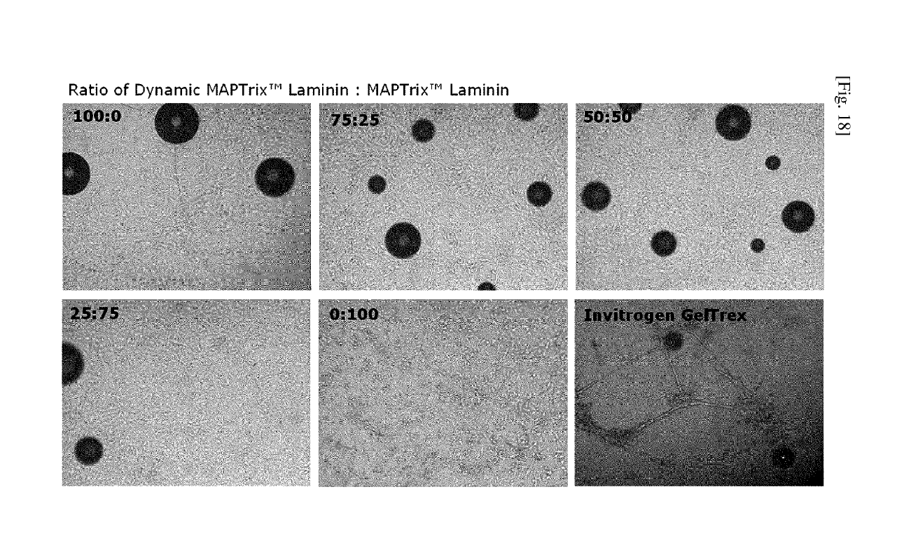

As seen in FIGS. 17a and 17b, cell shape or morphology were very similar to each other even though bFGF treated cells were more closer to tube formation.

Two examples showed MAPTrix™ GF based hydrogel can provide a synthetic microenvironment to present soluble factors.

### Example 9

### Fibroblast Cultured on a Hydrogel Formed from Dynamic MAPTrix™ Laminin

Enzyme sensitive MAPTrix™ HyGel gel solution was prepared as follows: Dynamic MAPTrix™ Laminin and MAPTrix™ Laminin were dissolved to a final concentration of 20 mg/ml, respectively, in PBS buffer solution (pH 7.4) and was mixed with 10 mg/ml 4 ARM and 8 ARM PEG-SG, (1:1 (v/v)). The ratio of Dynamic MAPTrix™ Laminin to MAPTrix™ Laminin was 100:0, 75:25, 50:50, 25:75 and 0:100. The prepared MAPTrix™ HyGel solutions were added to a 48-well plate (BD Biosciences) and allowed to gel at 37° C. for 2 hours. Invitrogen's GelTrex, reconstituted basement membrane, was used as a positive control.

Endothelial growth media (M199 media), supplemented with 10% fetal bovine serum (FBS) and endothelial cell growth supplement (ECGS, 30 μg/ml; BD Biosciences), was used to seed Hs27 dermal fibroblast cells.

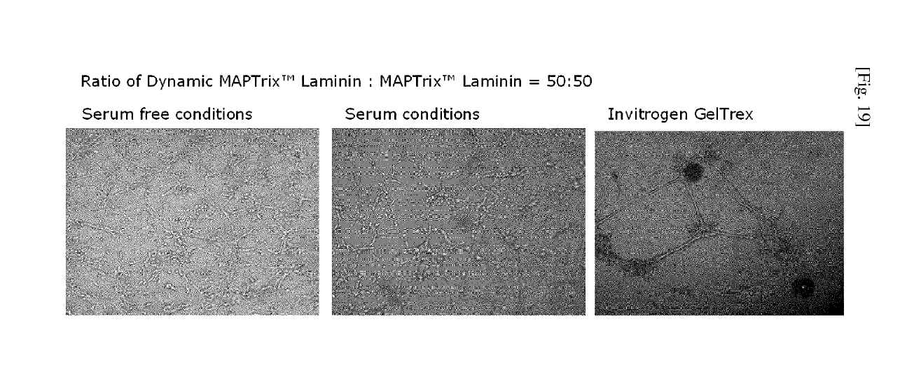

Hs27 dermal fibroblast cells were washed in serum-free M199 medium by centrifuging at 400 g for 1 min, and the washed Hs27 dermal fibroblast were resuspended in serum-free M199 medium and seeded onto each Dynamic MAPTrix™ hydrogel at a density of 3×10⁴ cells per well and incubated at 37° C. for 6 hours. The morphology of Hs27 dermal fibroblast was monitored and photographed with a phase contrast microscope (see FIG. 18).

Fibroblast cells formed a tube-like structure at a ratio of Dynamic MAPTrix™ Laminin to MAPTrix™ Laminin (75:25 and 50:50), similar to that of fibroblast cultured on GelTrex (FIG. 19). When FSB (10%) was added to the MAPTrix™ HyGel to generate a combinatorial signal, the cell morphology was more similar to that of cells on GelTrex.

It is evident that the dynamic properties of a substrate (i.e. controlled degradation by enzyme) together with combinatorial signals significantly affected the cellular behavior as demonstated in this EXMAPLE.

1. A synthetic microenvironment comprising a biomaterial composition presenting at least one or more ECM-derived peptide motifs that regulate cellular behavior such as cell adhesion, migration, growth or differentiation.

2. The synthetic microenvironment of claim 1, wherein a biomaterial composition for microenvironment comprising a mussel adhesive protein and a crosslinking agent.

3. The synthetic microenvironment of claim 2, wherein said mussel adhesive protein is functionalized with at least one or more extracellular matrix- or growth factor derived peptide motifs.

4. The synthetic microenvironment of claim 3, wherein said ECM derived peptide motif is selected from collagen, fibronectin, laminin, vitronectin, or cadherin, and said GF derived peptide motif is selected from fibroblast growth factor, transforming growth factor, epidermal growth factor, nerve growth factor, platelet derived growth factor, or vescular endothelial growth factor.

5. The synthetic microenvironment of claim 3, wherein said ECM or GF derived peptide motifs comprise a combination that activate at least two different cell surface receptors at the same time.

6. The synthetic microenvironment of claim 5, wherein said two different cell surface receptors are selected from integrins, syndecans, cadherins, dystroglycan, or growth factor receptors.

7. The synthetic microenvironment of claim 5, wherein said integrins are selected from α1β1, α2β1, α3β1, α4β1, α5β1, α6β1, αvβ3, or αvβ5.

8. The synthetic microenvironment of claim 5, wherein said syndecans are selected from syndecan-1, syndecan-2, syndecan-3 or syndecan-4.

9. The synthetic microenvironment of claim 5, wherein said growth factors are selected from fibroblast growth factor receptors, transforming growth factor receptor, epidermal growth factor receptor, nerve growth factor receptor, platelet derived growth factor receptor, or vascular endothelial growth factor receptor.

10. The synthetic microenvironment of claim 5, wherein said one cell surface receptor is selected from integrins and the other one cell surface receptor is selected from syndecans, cadherins, or dystroglycan.

11. The synthetic microenvironment of claim 5, wherein said one cell surface receptor is selected from integrins or heparin and the other once cell surface receptor is selected from growth factor receptors.

12. A method for preparing microenvironment array comprising:
(a) obtaining a crosslinkable ECM composition;
(b) placing a crosslinkable ECM composition on a solid support in a pattern; and
(c) crosslinking the ECM composition to obtain a synthetic microenvironment array,
wherein the crosslinkable ECM composition comprising a mussel adhesive protein functionalized with bioactive peptide and a crosslinkable agent.

13. An extracellular microenvironment surface regulating cellular behaviors, wherein said microenvironment surface presents at least one or more ECM- or GF-derived peptide motifs to regulate cellular behaviors by activating cell surface receptors to induce a combinatorial signaling in order to regulate cell adhesion, spreading, growth or differentiation.

14. The extracellular microenvironment surface of claim 13, wherein said microenvironment surface comprises mussel adhesive protein recombinantly functionalized with at least one ECM- or GF-derived peptide motif and at least matricryptic peptide motif.

15. The spatiotemporally controlled extracellular microenvironment surface of claim 13, wherein
the extracellular microenvironment surface is spatiotemporally controlled; and
said ECM- or GF-derived peptide motif is adjacent to said matricrptic peptide motif,
wherein an enzymatic digestion lead to the exposure of ECM or GF-derived peptide motif to cells to regulate cell adhesion, migration, growth or differentiation.

16. The extracellular microenvironment surface of claim 13 comprising mussel adhesive protein.

17. The extracellular microenvironment surface of claim 16, wherein the mussel adhesive protein is recombinantly functionalized with at least one ECM- or GF-derived peptide motif and at least one enzyme sensitive peptide motif, inducing combinatorial signaling to regulate cell adhesion, migration, growth or differentiation.

18. A synthetic extracellular microenvironment having the physical or mechanical cues mimics the physical or mechanical cues of a native extracellular microenvironment.

19. The synthetic extracellular microenvironment of claim 18, wherein said modulus of about 0.2 kPa to 2 kPa.

20. The synthetic extracellular microenvironment of claim 18, wherein said pore size of about 10 μm to about 100 μm.

21. A method for culturing and maintaining cells in vitro, comprising;
seeding at least one cell on a synthetic extracellular microenvironment,
wherein the extracellular microenvironment has biochemical and physical cue that is matched to the biomechical and physical cues of the tissue from which the cell is derived; and maintaining the cell in vitro.


SEQUENCE LISTING (116 sequences)

SEQ ID NO: 1 - protein, 10 aa, Artificial Sequence
  model peptide of the tandem repeat decapeptide derived from foot protein 1 (FP-1, Mytilus edulis)
AKPSYPPTYK

SEQ ID NO: 2 - protein, 20 aa, Artificial Sequence
  2 times repeated sequence derived from foot protein 1 (FP-1, Mytilus edulis)
AKPSYPPTYKAKPSYPPTYK

SEQ ID NO: 3 - protein, 60 aa, Artificial Sequence
  6 times repeated sequence derived from foot protein 1 (FP-1, Mytilus edulis)
AKPSYPPTYKAKPSYPPTYKAKPSYPPTYKAKPSYPPTYKAKPSYPPTYKAKPSYPPTYK

SEQ ID NO: 4 - protein, 39 aa, Artificial Sequence
  partial sequence of foot protein type 2 (FP-2, Mytilus californianus)
EVHACKPNPCKNNGRCYPDGKTGYKCKCVGGYSGPTCAC

SEQ ID NO: 5 - protein, 52 aa, Artificial Sequence
  Foot protein type 3 (FP-3, Mytilus edulis)
ADYYGPKYGPPRRYGGGNYNRYGGSRRYGGYKGWNNGWKRGRWGRKYYEFEF

SEQ ID NO: 6 - protein, 46 aa, Artificial Sequence
  Foot protein type 3 (FP-3, Mytilus galloprovincialis? mgfp-3A)
ADYYGPKYGPPRRYGGGNYNRYGRRYGGYKGWNNGWKRGRWGRKYY

SEQ ID NO: 7 - protein, 50 aa, Artificial Sequence
  Foot protein type 3 (FP-3, Mytilus edulis mefp-3F)
ADYYGPNYGPPRRYGGGNYNRYNGYGGGRRYGGYKGWNNGWNRGRRGKYW

SEQ ID NO: 8 - protein, 44 aa, Artificial Sequence
  Foot protein type 3 (FP-3, Mytilus californianus)
GAYKGPNYNYPWRYGGKYNGYKGYPRGYGWNKGWNKGRWGRKYY

SEQ ID NO: 9 - protein, 60 aa, Artificial Sequence
  partial sequence from foot protein type 4 (Mytilus californianus)
GHVHRHRVLHKHVHNHRVLHKHLHKHQVLHGHVHRHQVLHKHVHNHRVLHKHLHKHQVLH

SEQ ID NO: 10 - protein, 75 aa, Artificial Sequence
  Foot protein type5 (FP-5, Mytilus edulis)
SSEEYKGGYYPGNAYHYHSGGSYHGSGYHGGYKGKYYGKAKKYYYKYKNSGKYKYLKKARKYHRKGYKKYYGGSS

SEQ ID NO: 11 - protein, 76 aa, Artificial Sequence
  Foot protein 5 (FP-5, Mytilus edulis)
SSEEYKGGYYPGNTYHYHSGGSYHGSGYHGGYKGKYYGKAKKYYYKYKNSGKYKYLKKARKYHRKGYKKYYGGGSS

SEQ ID NO: 12 - protein, 71 aa, Artificial Sequence
  Foot protein 5 (FP-5, Mytilus coruscus)
YDDYSDGYYPGSAYNYPSGSHWHGHGYKGKYYGKGKKYYYKFKRTGKYKYLKKARKYHRKGYKKHYGGSSS

SEQ ID NO: 13 - protein, 76 aa, Artificial Sequence; identical to SEQ ID NO: 11
  mussel adhesive protein foot protein type5 from Mytilus galloprovincialis

SEQ ID NO: 14 - protein, 99 aa, Artificial Sequence
  mussel adhesive protein foot protein type 6
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 15 - protein, 194 aa, Artificial Sequence
  hybrid mussel adhesive protein (FP-151, MEFP-5 based)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 16 - protein, 196 aa, Artificial Sequence
  hybrid mussel adhesive protein (FP-151, MGFP-5 based)
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 17 - protein, 192 aa, Artificial Sequence
  hybrid mussel adhesive protein (FP-151, MCFP-5 based)
AKPSYPPTYKAKPSYPPTYKAKPSYPPTYKAKPSYPPTYKAKPSYPPTYKAKPSYPPTYKYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 18 - protein, 177 aa, Artificial Sequence
  hybrid mussel adhesive protein (FP-131)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKL

SEQ ID NO: 19 - protein, 180 aa, Artificial Sequence
  hybrid mussel adhesive protein (FP-251)
MEVHACKPNPCKNNGRCYPDGKTGYKCKCVGGYSGPTCACSSEEYKGGYYPGNSNHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKK

SEQ ID NO: 20 - protein, 6 aa, Artificial Sequence
  cell binding domain fragment of collagen type I (GLPGER)
GLPGER

SEQ ID NO: 21 - protein, 6 aa, Artificial Sequence
  cell binding domain fragment of collagen type I (KGHRGF)
KGHRGF

SEQ ID NO: 22 - protein, 6 aa, Artificial Sequence
  cell binding domain fragment of collagen type I (GFPGER)
GFPGER

SEQ ID NO: 23 - protein, 4 aa, Artificial Sequence
  cell binding domain fragment of collagen type I (DEGA)
DGEA

SEQ ID NO: 24 - protein, 15 aa, Artificial Sequence
  cell binding domain fragment of collagen type I (GTPGPQGIAGQRGVV)
GTPGPQGIAGQRGVV

SEQ ID NO: 25 - protein, 6 aa, Artificial Sequence
  cell binding domain fragment of collagen type I (GLSGER)
GLSGER

SEQ ID NO: 26 - protein, 6 aa, Artificial Sequence
  cell binding domain fragment of collagen type I (GASGER)
GASGER

SEQ ID NO: 27 - protein, 6 aa, Artificial Sequence
  cell binding domain fragment of collagen type I (GAPGER)
GAPGER

SEQ ID NO: 28 - protein, 12 aa, Artificial Sequence
  cell binding domain fragment of collagen type IV (TAGSCLRKFSTM)
TAGSCLRKFSTM

SEQ ID NO: 29 - protein, 13 aa, Artificial Sequence
  cell binding domain fragment of collagen type IV (GEFYFDLRLKGDK)
GEFYFDLRLKGDK

SEQ ID NO: 30 - protein, 15 aa, Artificial Sequence
  cell binding domain fragment of collagen type IV (TAIPSCPEGTVPLYS)
TAIPSCPEGTVPLYS

SEQ ID NO: 31 - protein, 7 aa, Artificial Sequence
  cell binding domain fragment of collagen type IV (MNYYSNS)
MNYYSNS

SEQ ID NO: 32 - protein, 12 aa, Artificial Sequence
  cell binding domain fragment of collagen type IV (ISRCQVCMKKRH)
ISRCQVCMKKRH

SEQ ID NO: 33 - protein, 6 aa, Artificial Sequence
  cell binding domain fragment of collagen type III (GLKGEN)
GLKGEN

SEQ ID NO: 34 - protein, 6 aa, Artificial Sequence
  cell binding domain fragment of collagen type III (GLPGEN)
GLPGEN

SEQ ID NO: 35 - protein, 6 aa, Artificial Sequence
  cell binding domain fragment of collagen type III (GLPGEA)
GLPGEA

SEQ ID NO: 36 - protein, 13 aa, Artificial Sequence
  cell binding domain fragment of laminin (RQVFQVAYIIIKA)
RQVFQVAYIIIKA

SEQ ID NO: 37 - protein, 5 aa, Artificial Sequence
  cell binding domain fragment of laminin (IKVAV)
IKVAV

SEQ ID NO: 38 - protein, 12 aa, Artificial Sequence
  cell binding domain fragment of laminin (NRWHSIYITRFG)
NRWHSIYITRFG

SEQ ID NO: 39 - protein, 12 aa, Artificial Sequence
  cell binding domain fragment of laminin (TWYKIAFQRNRK)
TWYKIAFQRNRK

SEQ ID NO: 40 - protein, 12 aa, Artificial Sequence
  cell binding domain fragment of laminin (RKRLQVQLSIRT)
RKRLQVQLSIRT

SEQ ID NO: 41 - protein, 12 aa, Artificial Sequence
  cell binding domain fragment of laminin (KNSFMALYLSKG)
KNSFMALYLSKG

SEQ ID NO: 42 - protein, 19 aa, Artificial Sequence
  cell binding domain fragment of laminin (DYATLQLQEGRLHFMFDLG)
DYATLQLQEGRLHFMFDLG

SEQ ID NO: 43 - protein, 6 aa, Artificial Sequence
  cell binding domain fragment of laminin (GIIFFL)
GIIFFL

SEQ ID NO: 44 - protein, 5 aa, Artificial Sequence
  cell binding domain fragment of laminin (YIGSR)
YIGSR

SEQ ID NO: 45 - protein, 7 aa, Artificial Sequence
  cell binding domain fragment of laminin (RYVVLPR)
RYVVLPR

SEQ ID NO: 46 - protein, 5 aa, Artificial Sequence
  cell binding domain fragment of laminin (PDSGR)
PDSGR

SEQ ID NO: 47 - protein, 12 aa, Artificial Sequence
  cell binding domain fragment of laminin (KAFDITYVRLKF)
KAFDITYVRLKF

SEQ ID NO: 48 - protein, 10 aa, Artificial Sequence
  cell binding domain fragment of laminin (RNIAEIIKDI)
RNIAEIIKDI

SEQ ID NO: 49 - protein, 6 aa, Artificial Sequence
  cell binding domain fragment of fibronectin (KLDAPT)
KLDAPT

SEQ ID NO: 50 - protein, 5 aa, Artificial Sequence
  cell binding domain fragment of fibronectin (PHSRN)
PHSRN

SEQ ID NO: 51 - protein, 3 aa, Artificial Sequence
  cell binding domain fragment of fibronectin (RGD)
RGD

SEQ ID NO: 52 - protein, 6 aa, Artificial Sequence
  cell binding domain fragment of fibronectin (GRGDSP)
GRGDSP

SEQ ID NO: 53 - protein, 20 aa, Artificial Sequence
  cell binding domain fragment of fibronectin (PHSRNSGSGSGSGSGRGDSP)
PHSRNSGSGSGSGSGRGDSP

SEQ ID NO: 54 - protein, 16 aa, Artificial Sequence
  cell binding domain fragment of fibronectin (YRVRVTPKEKTGPMKE)
YRVRVTPKEKTGPMKE

SEQ ID NO: 55 - protein, 7 aa, Artificial Sequence
  cell binding domain fragment of fibronectin (EDGIHEL)
EDGIHEL

SEQ ID NO: 56 - protein, 9 aa, Artificial Sequence
  cell binding domain fragment of fibronectin (SPPRRARVT)
SPPRRARVT

SEQ ID NO: 57 - protein, 8 aa, Artificial Sequence
  cell binding domain fragment of fibronectin (WQPPRARI)
WQPPRARI

SEQ ID NO: 58 - protein, 15 aa, Artificial Sequence
  cell binding domain fragment of fibronectin (KNNQKSEPLIGRKKT)
KNNQKSEPLIGRKKT

SEQ ID NO: 59 - protein, 8 aa, Artificial Sequence
  cell binding domain fragment of fibronectin (EILDVPST)
EILDVPST

SEQ ID NO: 60 - protein, 5 aa, Artificial Sequence
  cell binding domain fragment of fibronectin (IDAPS)
IDAPS

SEQ ID NO: 61 - protein, 4 aa, Artificial Sequence
  cell binding domain fragment of fibronectin (REDV)
REDV

SEQ ID NO: 62 - protein, 4 aa, Artificial Sequence
  cell binding domain fragment of fibronectin (LEDV)
LEDV

SEQ ID NO: 63 - protein, 9 aa, Artificial Sequence
  cell binding domain fragment of vitronectin (RGDV)
FRHRNRKGY

SEQ ID NO: 64 - protein, 9 aa, Artificial Sequence
  cell binding domain fragment of vitronectin (RGDF)
FRHRNRKGY

SEQ ID NO: 65 - protein, 8 aa, Artificial Sequence
  enzyme cleavage site from MMP (GPQGIAGQ)
GPQGIAGQ

SEQ ID NO: 66 - protein, 8 aa, Artificial Sequence
  enzyme cleavage site from MMP (GPQGIASQ)
GPQGIASQ

SEQ ID NO: 67 - protein, 8 aa, Artificial Sequence
  enzyme cleavage site from MMP (GPQGIFGQ)
GPQGIWGQ

SEQ ID NO: 68 - protein, 8 aa, Artificial Sequence
  enzyme cleavage site from MMP (GPQGIWGQ)
GPQGIWGQ

SEQ ID NO: 69 - protein, 8 aa, Artificial Sequence
  enzyme cleavage site from MMP (GPVGIAGQ)
GPVGIAGQ

SEQ ID NO: 70 - protein, 8 aa, Artificial Sequence
  enzyme cleavage site from MMP (GPQGVAGQ)
GPQGVAGQ

SEQ ID NO: 71 - protein, 8 aa, Artificial Sequence
  enzyme cleavage site from MMP (GPQGRAGQ)
GPQGRAGQ

SEQ ID NO: 72 - protein, 4 aa, Artificial Sequence
  enzyme cleavage site from collagenase (LGPA)
LGPA

SEQ ID NO: 73 - protein, 4 aa, Artificial Sequence
  enzyme cleavage site from collagenase (APGL)
APGL

SEQ ID NO: 74 - protein, 7 aa, Artificial Sequence
  enzyme cleavage site from factor XIIIa (NQEQVSP)
NQEQVSP

SEQ ID NO: 75 - protein, 7 aa, Artificial Sequence
  enzyme cleavage site from elastase (AAAAAAAA)
NQEQVSP

SEQ ID NO: 76 - protein, 4 aa, Artificial Sequence
  enzyme cleavage site from plasmin (YKNR)
YKNR

SEQ ID NO: 77 - protein, 6 aa, Artificial Sequence
  enzyme cleavage site from plasmin (NNRDNT)
NNRDNT

SEQ ID NO: 78 - protein, 7 aa, Artificial Sequence
  enzyme cleavage site from plasmin (YNRVSED)
YNRVSED

SEQ ID NO: 79 - protein, 6 aa, Artificial Sequence
  enzyme cleavage site from plasmin (LIKMKP)
LIKMKP

SEQ ID NO: 80 - protein, 3 aa, Artificial Sequence
  enzyme cleavage site from plasmin (VRN)
VRN

SEQ ID NO: 81 - protein, 6 aa, Artificial Sequence
  enzyme cleavage site from thrombin (GLVPRG)
GLVPRG

SEQ ID NO: 82 - protein, 33 aa, Artificial Sequence
  matricryptic mussel adhesive protein (AKPSYPPTYKAKPSYPPTYKIKVAVGPQGIAGQ)
AKPSYPPTYKAKPSYPPTYKIKVAVGPQGIAGQ

SEQ ID NO: 83 - protein, 34 aa, Artificial Sequence
  matricryptic mussel adhesive protein (AKPSYPPTYKAKPSYPPTYKGFPGERGPQGIAGQ )
AKPSYPPTYKAKPSYPPTYKGFPGERGPQGIAGQ

SEQ ID NO: 84 - protein, 34 aa, Artificial Sequence
  matricryptic mussel adhesive protein (AKPSYPPTYKAKPSYPPTYKGRGDSPGPQGIAGQ)
AKPSYPPTYKAKPSYPPTYKGRGDSPGPQGIAGQ

SEQ ID NO: 85 - protein, 39 aa, Artificial Sequence
  matricryptic mussel adhesive protein (AKPSYPPTYKAKPSYPPTYKGRGDSPIKVAVGPQGIAGQ)
AKPSYPPTYKAKPSYPPTYKGRGDSPIKVAVGPQGIAGQ

SEQ ID NO: 86 - protein, 47 aa, Artificial Sequence
  matricryptic mussel adhesive protein (AKPSYPPTYKAKPSYPPTYKIKVAVGPQGIAGQGFPGERGPQGIWGQ)
AKPSYPPTYKAKPSYPPTYKIKVAVGPQGIAGQGFPGERGPQGIWGQ

SEQ ID NO: 87 - protein, 47 aa, Artificial Sequence
  matricryptic mussel adhesive protein (AKPSYPPTYKAKPSYPPTYKIKVAVGPQGIAGQGRGDSPGPQGIWGQ)
AKPSYPPTYKAKPSYPPTYKIKVAVGPQGIAGQGRGDSPGPQGIWGQ

SEQ ID NO: 88 - protein, 207 aa, Artificial Sequence
  matricryptic mussel adhesive protein (IKVAV-GPQGIAGQ)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSIKVAVGPQGIAGQAKPSYPPTYKAKPSYPPTYKAKPSYPPTYKAKPSYPPTYKAKPSYPPTYKAKPSYPPTYK

SEQ ID NO: 89 - protein, 207 aa, Artificial Sequence
  matricryptic mussel adhesive protein (IKVAV-GPQGIAGQ)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYKIKVAVGPQGIAGQ

SEQ ID NO: 90 - protein, 313 aa, Artificial Sequence
  Hybrid mussel adhesive protein (FP-13151)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKLSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 91 - protein, 16 aa, Artificial Sequence
  active domain from acidic fibroblast growth factor (FGF-1) (TGQYLAMDTDGLLYGS)
TGQYLAMDTDGLLYGS

SEQ ID NO: 92 - protein, 14 aa, Artificial Sequence
  active domain from acidic fibroblast growth factor (FGF-1) (WFVGLKKNG SCKRG)
WFVGLKKNGSCKRG

SEQ ID NO: 93 - protein, 11 aa, Artificial Sequence
  active domain from basic fibroblast growth factor (FGF-2) (HFKDPKRLYCK)
HFKDPKRLYCK

SEQ ID NO: 94 - protein, 8 aa, Artificial Sequence
  active domain from basic fibroblast growth factor (FGF-2) (FLPMSAKS)
FLPMSAKS

SEQ ID NO: 95 - protein, 10 aa, Artificial Sequence
  active domain from basic fibroblast growth factor (FGF-2) (KTGPGQKAIL)
KTGPGQKAIL

SEQ ID NO: 96 - protein, 16 aa, Artificial Sequence
  active domain from basic fibroblast growth factor (FGF-2) (ANRYLAMKEDGRLLAS)
ANRYLAMKEDGRLLAS

SEQ ID NO: 97 - protein, 14 aa, Artificial Sequence
  active domain from basic fibroblast growth factor (FGF-2) (WYVALKRTGQYKLG)
WYVALKRTGQYKLG

SEQ ID NO: 98 - protein, 15 aa, Artificial Sequence
  heparin binding motif derived from collagen (GDLGRPGRKGRPGPP)
GDLGRPGRKGRPGPP

SEQ ID NO: 99 - protein, 15 aa, Artificial Sequence
  heparin binding motif derived from fibronectin (ATETTITISWRTKTE)
ATETTITISWRTKTE

SEQ ID NO: 100 - protein, 12 aa, Artificial Sequence
  heparin binding motif derived from laminin (TLFLAHGRLVFM)
TLFLAHGRLVFM

SEQ ID NO: 101 - protein, 9 aa, Artificial Sequence
  heparin binding motif derived from vitronectin (FRHRNRKGY)
FRHRNRKGY

SEQ ID NO: 102 - protein, 4 aa, Artificial Sequence
  heparin binding motif derived from bone sialoprotein (KRSR)
KRSR

SEQ ID NO: 103 - protein, 7 aa, Artificial Sequence
  heparin binding motif derived from bone sialoprotein (FHRRIKA)
FHRRIKA

SEQ ID NO: 104 - protein, 3 aa, Artificial Sequence
  cell binding motif derived from cadherin (HAV)
HAV

SEQ ID NO: 105 - protein, 6 aa, Artificial Sequence
  cell binding motif derived from cadherin (ADTPPV)
ADTPPV

SEQ ID NO: 106 - protein, 5 aa, Artificial Sequence
  cell binding motif derived from cadherin (DQNDN)
DQNDN

SEQ ID NO: 107 - protein, 16 aa, Artificial Sequence
  active domain from fibroblast growth factor-3 (FGF-3) (SGRYLAMNKRGRLYAS)
SGRYLAMNKRGRLYAS

SEQ ID NO: 108 - protein, 16 aa, Artificial Sequence
  active domain from fibroblast growth factor-9 (FGF-9) (SGLYLGMNEKGELYGS)
SGLYLGMNEKGELYGS

SEQ ID NO: 109 - protein, 16 aa, Artificial Sequence
  active domain from fibroblast growth factor-10 (FGF-10) (SNYYLAMNKKGKLYGS)
SNYYLAMNKKGKLYGS

SEQ ID NO: 110 - protein, 16 aa, Artificial Sequence
  active domain from fibroblast growth factor-17 (FGF-17) (SEKYICMNKRGKLIGK)
SEKYICMNKRGKLIGK

SEQ ID NO: 111 - protein, 12 aa, Artificial Sequence
  active domain from transforming growth factor alpha (TGF-alpha) (HADLLAVVAASQ)
HADLLAVVAASQ

SEQ ID NO: 112 - protein, 8 aa, Artificial Sequence
  active domain from transforming growth factor beta (TGF-beta) (KVLALYNK)
KVLALYNK

SEQ ID NO: 113 - protein, 12 aa, Artificial Sequence
  active domain from epidermal growth factor (EGF) (CMHIESLDSYTC)
CMHIESLDSYTC

SEQ ID NO: 114 - protein, 17 aa, Artificial Sequence
  active domain from nerve growth factor (NGF) (PEAHWTKLQHSLDTALR)
PEAHWTKLQHSLDTALR

SEQ ID NO: 115 - protein, 11 aa, Artificial Sequence
  active domain from platelet derived growth factor (PDGF) (SVLYTAVQPNE)
SVLYTAVQPNE

SEQ ID NO: 116 - protein, 15 aa, Artificial Sequence
  active domain from vascular endothelial growth factor (VEGF) (KLTWQELYQLKYKGI)
KLTWQELYQLKYKGI